SEQUENCE LISTING (28 sequences)

SEQ ID NO: 1 - DNA, 1256 bp, Staphylococcus aureus
tagcaataccttttcctctagctgaagcatcgacataaatagaatgttcgattgtatataggtatgctggccaaggtctaaatgaaccgaacgtcgcaaaccctaagacacttccattttcctcaaatacaaagataggctcatgcttacgttgtttcgtttcaaaccatgcgacacgttcgtctatggtttgtggttcataagtataaacagctgtagtattgataatggcatcattgtatatcgctaatatagcgtttaaatcctcttttttagcgtatctaatcatatcaattcccccttagtaattattaaaagcgtttcgttatttgaatgcaaatatgtgtaatgaaatctaacgtaaaagtatacatgtaaattttatagtataaaatgaattgctatgagtcattttgaaattaatggtatactatatgaaatgttaacaggcattgtgaaatgtataaaaggagccttaacgtatgaaaaaatggacaaatcgattaatgacaatcgctggtgtggtacttatcctagtggcagcatatttgtttgctaaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaataatctattacgctaatggatgaatatattgagtggaaaacagtcttgattgcgagactgttttttgtttggtatgaggtagcaatgacgacgtgtcattggtggagattgtaaaaatacataataaaaagaagcggcaatgtataccgctccttt

SEQ ID NO: 2 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 3 - DNA, 735 bp, Staphylococcus aureus
atgagaatgaagcgatttttaactattgtacaaattttattggttgtaattattatcatttttggttacaaaattgttcaaacatatattgaagacaagcaagaacgcgcaaattatgagaaattacaacaaaaatttcaaatgctgatgagcaaacatcaagcacatgtgagaccacaatttgaatcacttgaaaaaataaataaagacattgttggatggataaaattatcaggaacatcattaaattatccagtactacaaggtaagacaaatcacgattatttaaatttagattttgagcgagaacatcgacgtaaaggtagtatttttatggattttagaaatgaattgaagaatttaaatcataatactattttatacgggcaccatgtcggtgataatacgatgtttgatgtgttagaagattatttaaagcaatcgttttatgaaaaacacaagataattgaatttgacaataaatatggtaaatatcaattgcaagtatttagtgcatataaaactactactaaagataattacatacgtacagattttgaaaatgatcaagattatcaacaatttttagatgaaacaaaacgtaaatctgtaattaattcagatgttaatgtaacggtaaaagatagaataatgactttatcaacgtgcgaagatgcatatagtgaaacaacgaaaagaattgttgttgtcgcaaaaataattaaggtaagttaa

SEQ ID NO: 4 - protein, 244 aa, Staphylococcus aureus
MRMKRFLTIVQILLVVIIIIFGYKIVQTYIEDKQERANYEKLQQKFQMLMSKHQAHVRPQFESLEKINKDIVGWIKLSGTSLNYPVLQGKTNHDYLNLDFEREHRRKGSIFMDFRNELKNLNHNTILYGHHVGDNTMFDVLEDYLKQSFYEKHKIIEFDNKYGKYQLQVFSAYKTTTKDNYIRTDFENDQDYQQFLDETKRKSVINSDVNVTVKDRIMTLSTCEDAYSETTKRIVVVAKIIKVS

SEQ ID NO: 5 - protein, 5 aa, Artificial Sequence
  Description of Artificial Sequence Synthetic peptide
XPXXG

SEQ ID NO: 6 - protein, 5 aa, Artificial Sequence
  Description of Artificial Sequence Synthetic peptide
LPXTG

SEQ ID NO: 7 - protein, 5 aa, Artificial Sequence
  Description of Artificial Sequence Synthetic peptide
NPXTX

SEQ ID NO: 8 - protein, 5 aa, Staphylococcus aureus
NPQTN

SEQ ID NO: 9 - protein, 9 aa, Artificial Sequence
  Description of Artificial Sequence Synthetic peptide
RELPKTGKR

SEQ ID NO: 10 - protein, 16 aa, Artificial Sequence
  Description of Artificial Sequence Synthetic peptide
GGGGGRRNRRTSKLMR

SEQ ID NO: 11 - protein, 14 aa, Artificial Sequence
  Description of Artificial Sequence Synthetic peptide
GFPLPXTGHHHHHH

SEQ ID NO: 12 - protein, 14 aa, Artificial Sequence
  Description of Artificial Sequence Synthetic peptide
GSTLPXTGHHHHHH

SEQ ID NO: 13 - protein, 23 aa, Artificial Sequence
  Description of Artificial Sequence Synthetic peptide
RELPKTGGGGGRRNRRTSKLMLR

SEQ ID NO: 14 - protein, 17 aa, Artificial Sequence
  Description of Artificial Sequence Synthetic peptide
GGGGGRRNRRTSKLMLR

SEQ ID NO: 15 - protein, 191 aa, Artificial Sequence
  Description of Artificial Sequence Synthetic polypeptide
MQFTSLLLAVIFLISLVSIDGGGGGMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 16 - protein, 20 aa, Artificial Sequence
  Description of Artificial Sequence Synthetic peptide
MQFTSLLLAVIFLISLVSID

SEQ ID NO: 17 - protein, 5 aa, Artificial Sequence
  Description of Artificial Sequence Synthetic peptide
GGGGG

SEQ ID NO: 18 - protein, 166 aa, Artificial Sequence
  Description of Artificial Sequence Synthetic polypeptide
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 19 - protein, 5 aa, Artificial Sequence
  Description of Artificial Sequence Synthetic peptide
LPXTG

SEQ ID NO: 20 - protein, 171 aa, Artificial Sequence
  Description of Artificial Sequence Synthetic polypeptide
GGGGGMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 21 - protein, 175 aa, Artificial Sequence
  Description of Artificial Sequence Synthetic polypeptide
LPXTGGGGGMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 22 - protein, 5 aa, Artificial Sequence
  Description of Artificial Sequence Synthetic peptide
XPXXG

SEQ ID NO: 23 - protein, 5 aa, Artificial Sequence
  Description of Artificial Sequence Synthetic peptide
GGGGG

SEQ ID NO: 24 - protein, 9 aa, Artificial Sequence
  Description of Artificial Sequence Synthetic peptide
LPXTGGGGG

SEQ ID NO: 25 - protein, 4 aa, Artificial Sequence
  Description of Artificial Sequence Synthetic peptide
GGGG

SEQ ID NO: 26 - protein, 6 aa, Artificial Sequence
  Description of Artificial Sequence Synthetic 6xHis tag
HHHHHH

SEQ ID NO: 27 - protein, 10 aa, Artificial Sequence
  Description of Artificial Sequence Synthetic peptide
GGGGGGGGGG

SEQ ID NO: 28 - protein, 17 aa, Artificial Sequence
  Description of Artificial Sequence Synthetic peptide
GGGGGRRNRRTSKLMLR

(19) United States
(12) Patent Application Publication
(10) Pub. No.: US 2012/0282670 A1
(43) Pub. Date: Nov. 8, 2012

(54) COMPOSITIONS AND METHODS FOR ENHANCING PRODUCTION OF A BIOLOGICAL PRODUCT

(75) Inventor: Anthony Rossomando, Cambridge, MA (US)

(73) Assignee: Alnylam Pharmaceuticals, Inc., Cambridge, MA (US)

(21) Appl. No.: 13/505,813

(22) PCT Filed: Nov. 4, 2010
(86) PCT No.: PCT/US10/55355

§ 371 Date: Jul. 23, 2012

Related U.S. Application Data
(60) Provisional application No. 61/258,149, filed on Nov. 4, 2009.

(51) Int. Cl. C12N 9/96; C07H 21/04

(52) U.S. Cl. 435/188; 536/23.2

(57) ABSTRACT

Provided herein are methods, nucleic acids, polypeptides, compositions, and kits relating to the conjugation of a heterologous polypeptide to a molecule of interest during production of the polypeptide in cell culture. In various embodiments, the heterologous polypeptide is linked to a sortase ligation sequence and the molecule of interest is linked to a complementary sortase ligation sequence, such that expression of the heterologous protein in the presence of the molecule of interest and cells expressing a surface-associated sortase with the sortase catalytic domain exposed to the extracellular medium results in ligation of the heterologous polypeptide to the molecule of interest to form a conjugated polypeptide.

### CROSS-REFERENCE TO RELATED APPLICATIONS

This Application claims the benefit under 35 U.S.C §119(e) of U.S. Provisional Application No. 61/258,149 filed Nov. 4, 2009, which is herein incorporated by reference in it's entirety.

### FIELD OF THE INVENTION

The invention relates generally to the field of bioprocessing and more particularly to methods for conjugating a heterologous polypeptide to a molecule of interest in cell culture. The heterologous polypeptide is expressed as a secreted fusion protein comprising a sortase conjugation sequence in the presence of host cells having cell surface sortase activity, and addition of a conjugation substrate comprising the molecule of interest and a complementary sortase conjugation sequence results in selective conjugation and formation of a conjugated polypeptide. The invention also relates to molecules, reagents, cells, and kits useful for carrying out such methods and conjugated polypeptides produced by such methods.

### BACKGROUND OF THE INVENTION

Protein conjugation by chemical ligation (direct covalent coupling) is a fundamental and widely used tool of protein engineering. However, ligation procedures have numerous drawbacks, including lack of specificity due to the presence of multiple reactive sites within a target protein, the need for organic solvents and other reagents that can adversely effect the structure and/or activity of proteins, and need for time-consuming additional processing steps for carrying out the ligation and the subsequent isolation of the conjugate. Accordingly, there is a need in the art for alternative methods that allow a wide range of molecules to be selectively ligated to a polypeptide.

### SUMMARY OF THE INVENTION

Provided herein are methods, compositions, kits and the like relating to the conjugation of a heterologous polypeptide to a molecule of interest during production of the polypeptide in cell culture. In various embodiments, the heterologous polypeptide is linked to a sortase ligation sequence and the molecule of interest is linked to a complementary sortase ligation sequence, such that expression of the heterologous protein in the presence of the molecule of interest and cells expressing a surface-associated sortase with the sortase catalytic domain exposed to the extracellular medium results in ligation of the heterologous polypeptide to the molecule of interest to form a conjugated polypeptide.

In one aspect, an isolated nucleic acid is provided which encodes a polypeptide comprising a eukaryotic signal sequence, a soluble sortase, and a transmembrane domain, wherein the signal sequence is capable of targeting the polypeptide for secretion by a eukaryotic host cell and the transmembrane domain is capable of anchoring the polypeptide in the plasma membrane of the host cell with the sortase exposed to the extracellular medium.

In some embodiments, the signal sequence is capable of being cleaved from the polypeptide by a native enzyme of the eukaryotic host cell.

In one embodiment, the transmembrane domain is located N-terminal of the sortase. In another embodiment, the transmembrane domain is located C-terminal of the sortase.

In some embodiments, the sortase has sortase A catalytic activity. In further embodiments, the sortase is sortase A of S. aureus, or a catalytically active fragment, derivative, or variant thereof. For example, in one embodiment, the sortase comprises residues 60-206 of sortase A of S. aureus. In further embodiments, the sortase has sortase B catalytic activity. In further embodiments, the sortase is sortase B of S. aureus, or a catalytically active fragment, derivative, or variant thereof. For example, in one embodiment, the sortase comprises residues 30-229 of sortase B of S. aureus

In some embodiments, the transmembrane domain is capable of anchoring the polypeptide in the plasma membrane in a type II orientation. In some embodiments, the transmembrane domain is located N-terminal of the sortase having sortase A catalytic activity.

In further embodiments, the transmembrane domain is capable of anchoring the polypeptide in the plasma membrane with a type I orientation. In some embodiments, the transmembrane domain is located C-terminal of the sortase having sortase B catalytic activity.

In some embodiments, the nucleotide sequence is operably linked to an expression control sequence, such as a eukaryotic promoter.

In some embodiments, the nucleic acid further encodes an affinity tag.

In further embodiments, the nucleic acid further encodes a spacer peptide. In some embodiments, the spacer peptide is located between the soluble sortase and the transmembrane domain.

In another aspect, an expression vector is provided comprising a nucleotide sequence encoding a fusion protein, the fusion protein comprising a heterologous polypeptide, a eukaryotic signal sequence capable of targeting the fusion protein for secretion by a eukaryotic host cell, and a sortase ligation sequence.

In a further aspect, a eukaryotic cell is provided which expresses a nucleic acid of the invention.

In an additional aspect, a recombinant polypeptide is provided comprising a eukaryotic signal sequence, a soluble sortase, and a transmembrane domain, wherein the signal sequence is capable of targeting the polypeptide for secretion by a eukaryotic host cell and the transmembrane domain is capable of anchoring the polypeptide in the plasma membrane of the host cell with the sortase exposed to the extracellular medium.

In another aspect, a recombinant polypeptide is provided comprising a eukaryotic signal sequence, a heterologous polypeptide, and a sortase ligation sequence, wherein the signal sequence is capable of targeting the polypeptide for secretion by a eukaryotic host cell.

In further embodiments, the recombinant polypeptide further comprises an affinity tag.

In further embodiments, the recombinant polypeptide further comprises a spacer peptide. In some embodiments, the spacer peptide is located between the soluble sortase and the transmembrane domain.

In some embodiments, the sortase ligation sequence comprises a sortase recognition sequence. In some embodiments, the sortase ligation sequence is located C-terminal of the heterologous polypeptide.

In some embodiments, the sortase recognition sequence is a sortase A recognition sequence having the consensus sequence X₁PX₂X₃G, wherein X₁ is Leu, Ile, Val or Met, P is Pro, X₂ is any amino acid, X₃ is Ser, Thr or Ala, and G is Gly (SEQ ID NO:5). In further embodiments, X₂ is Asp, Glu, Ala, Gln, Lys or Met (SEQ ID NO:22).

In some embodiments, the sortase recognition sequence is a sortase A recognition sequence having the consensus sequence LPXTG, wherein L is Leu, P is Pro, X is any amino acid, T is Thr, and G is Gly (SEQ ID NO:6).

In some embodiments, the sortase recognition sequence is a sortase B recognition sequence having the consensus sequence NPX₁TX₂, wherein N is Asn, P is Pro, X₁ is Gln or Lys, T is Thr, and X₂ is Asp or Gly (SEQ ID NO:7). In further embodiments, the sortase B recognition sequence is NPQTN (SEQ ID NO:8).

In some embodiments, the sortase ligation sequence is an polyglycine sequence. In some embodiments, the sortase ligation sequence comprises 1, 2, 3, 4 or 5 glycine residues. In some embodiments, the sortase ligation sequence is located N-terminal of the heterologous polypeptide. In other embodiments, the sortase ligation sequence is located C-terminal of a signal sequence, and N-terminal of the heterologous polypeptide.

In an additional aspect, an expression vector is provided comprising a nucleotide sequence encoding a fusion protein, the fusion protein comprising a heterologous polypeptide, a eukaryotic signal sequence capable of targeting the fusion protein for secretion by a eukaryotic host cell, and a sortase ligation sequence.

In yet an additional aspect, a method for producing a conjugated polypeptide is provided, comprising:

expressing a first nucleotide sequence encoding a first fusion protein in a cultured host cell, the first fusion protein comprising a first eukaryotic signal sequence, a transmembrane domain and a soluble sortase, wherein the first signal sequence targets the first fusion protein for secretion by the host cell and the transmembrane domain anchors the first fusion protein in the plasma membrane of the cell with the sortase exposed to the extracellular medium;

expressing a second nucleotide sequence encoding a second fusion protein in a cultured host cell, the second fusion protein comprising a second eukaryotic signal sequence, a heterologous polypeptide, and a first sortase ligation sequence, wherein the second signal sequence targets the second fusion protein for secretion by the host cell;

contacting the cell with a conjugation substrate comprising a second sortase ligation sequence and a molecule of interest, wherein one of the first or second sortase ligation sequences comprises a sortase recognition sequence and the other of the first or second sortase ligation sequences comprises a polyglycine sequence;

maintaining the cell under conditions which allow the sortase to cleave the sortase recognition sequence and ligate the cleaved sortase recognition sequence to the polyglycine sequence to form a conjugated polypeptide; and

isolating the conjugated polypeptide.

In some embodiments, the conjugation includes formation of an amide bond between a C-terminal carboxyl group of the cleaved sortase recognition sequence and an N-terminal amino group of the polyglycine sequence.

In some embodiments, the first sortase ligation sequence comprises the sortase recognition sequence and the second sortase ligation sequence comprises the polyglycine sequence. In further embodiments, the sortase recognition sequence is located C-terminal of the heterologous polypeptide.

In some embodiments, the second fusion protein further comprises an affinity tag located C-terminal of the sortase ligation sequence, the affinity tag being cleaved from the conjugated polypeptide.

In some embodiments, the second fusion protein further comprises an affinity tag located N-terminal of the sortase ligation sequence, the affinity tag being retained in the conjugated polypeptide.

In some embodiments, the conjugation substrate further comprises an affinity tag located C-terminal of the second sortase ligation sequence, the affinity tag being retained in the conjugated polypeptide.

In some embodiments, the first sortase ligation sequence comprises the polyglycine sequence and the second sortase ligation sequence comprises the sortase recognition sequence. In further embodiments, the second eukaryotic signal sequence is at the N-terminus of the second fusion protein and the polyglycine sequence is located C-terminal of the affinity tag. In yet further embodiments, the second eukaryotic signal sequence is capable of being cleaved by a host cell enzyme, wherein the polyglycine sequence is located at the N-terminus of the second fusion protein upon cleavage of the second eukaryotic signal sequence.

In other embodiments, the sortase recognition sequence is located C-terminal of the molecule of interest. In further embodiments, the conjugation substrate further comprises an affinity tag located N-terminal of the second sortase ligation sequence, the affinity tag being retained in the conjugated polypeptide. In yet further embodiments, the second fusion protein further comprises an affinity tag located C-terminal of the first sortase ligation sequence, the affinity tag being retained in the conjugated polypeptide.

In some embodiments, the sortase recognition sequence is a sortase A recognition sequence having the consensus sequence X₁PX₂X₃G, wherein X₁ is Leu, Ile, Val or Met, P is Pro, X₂ is any amino acid, X₃ is Ser, Thr or Ala, and G is Gly (SEQ ID NO:5). In further embodiments, X2 is Asp, Glu, Ala, Gln, Lys or Met (SEQ ID NO:22).

In some embodiments, the sortase recognition sequence is a sortase A recognition sequence having the consensus sequence LPXTG (SEQ ID NO:6), wherein L is Leu, P is Pro, X is any amino acid, T is Thr, and G is Gly.

In some embodiments, the sortase recognition sequence is a sortase B recognition sequence having the consensus sequence NPX₁TX₂, wherein N is Asn, P is Pro, X₁ is Gln or Lys, T is Thr, and X₂ is Asp or Gly (SEQ ID NO:7). In further embodiments, the sortase B recognition sequence is NPQTN (SEQ ID NO:8).

In some embodiments, the polyglycine sequence comprises 1, 2, 3, 4 or 5 glycine residues. In further embodiments, the first fusion protein further comprises a spacer peptide. In yet further embodiments, the spacer peptide is located between the sortase and the transmembrane domain.

In some embodiments, the second fusion protein further comprises a spacer peptide. In further embodiments, the spacer peptide is located between the heterologous polypeptide and the first sortase ligation sequence.

In some embodiments, the signal sequences of the first and/or second fusion proteins are capable of being cleaved by a host cell enzyme.

In some embodiments, the conjugation substrate is of the formula:

S-L-R

wherein S is a sortase ligation sequence, L is an optional linker and R is a molecule of interest.

In some embodiments, R or L comprises a water-soluble, non-peptidic polymer with an average molecular weight of about 200 to about 100,000 Daltons. In further embodiments, the polymer is a poly(ethylene glycol) (PEG) or a methoxypoly(ethylene glycol) (mPEG).

In further embodiments, R is selected from the group consisting of: silane, fluorescein, rhodamine, FITC and biotin.

In some embodiments, L is a hydrolytically stable linker. In further embodiments, L comprises at least 3 contiguous saturated carbon atoms.

In another aspect, a composition is provided comprising a conjugation substrate of the formula S-L-R, where S is the second sortase ligation sequence, L is an optional linker and R is the molecule of interest.

In some embodiments, the composition comprises a conjugation substrate of the general structure:

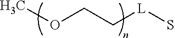

wherein n is 1 to 2,500, L is an optional linker, and S is a sortase ligation sequence.

The details of one or more embodiments of the invention are set forth in the description below. Other features, objects, and advantages of the invention will be apparent from the description and the drawings, and from the claims.

### DESCRIPTION OF THE FIGURES

FIG. 1 is a schematic representation of producing conjugated polypeptides. LPXTG, (SEQ ID NO:6)

FIG. 2 is a schematic representation of a surface exposed sortase construct.

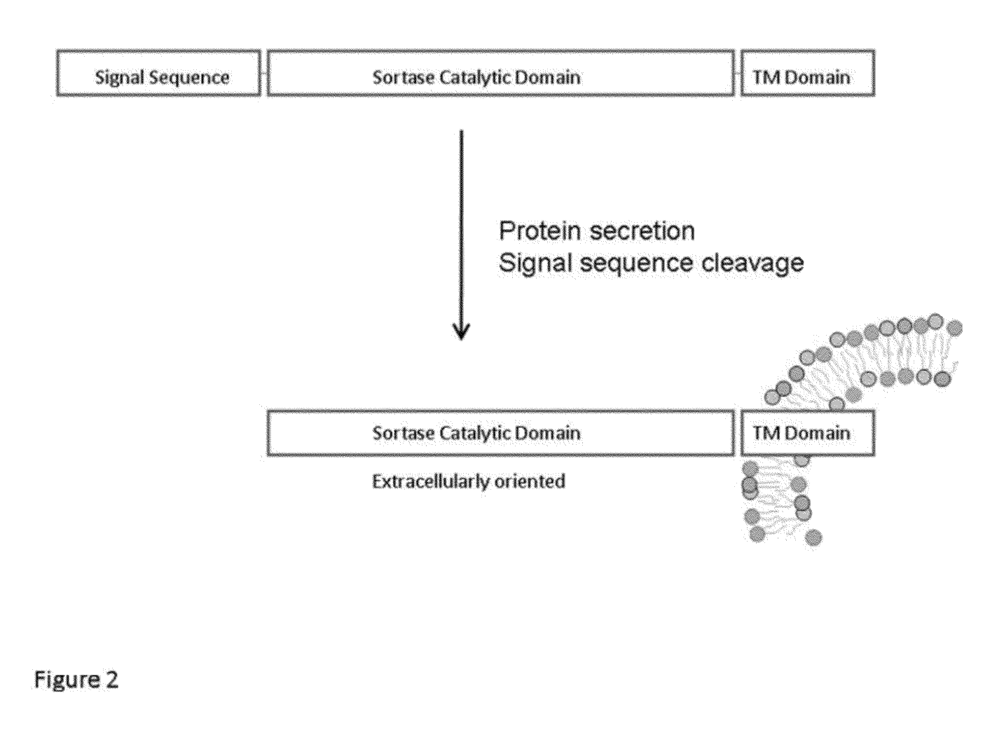

FIG. 3 depicts C-terminal conjugation of proteins; LPXTG (SEQ ID NO:6); GGGGG, (SEQ ID NO:23); LPXTGGGGGG (SEQ ID NO:24).

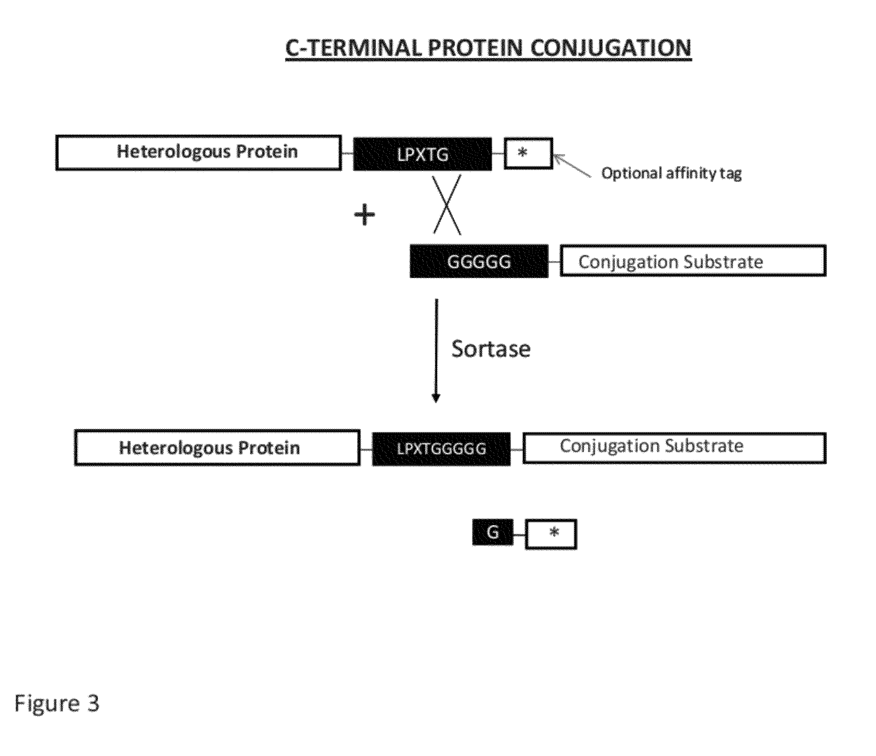

FIG. 4 depcits a N-terminal conjugation of proteins; LPXTG (SEQ ID NO:6); GGGGG, (SEQ ID NO:23); LPXTGGGGGG (SEQ ID NO:24)

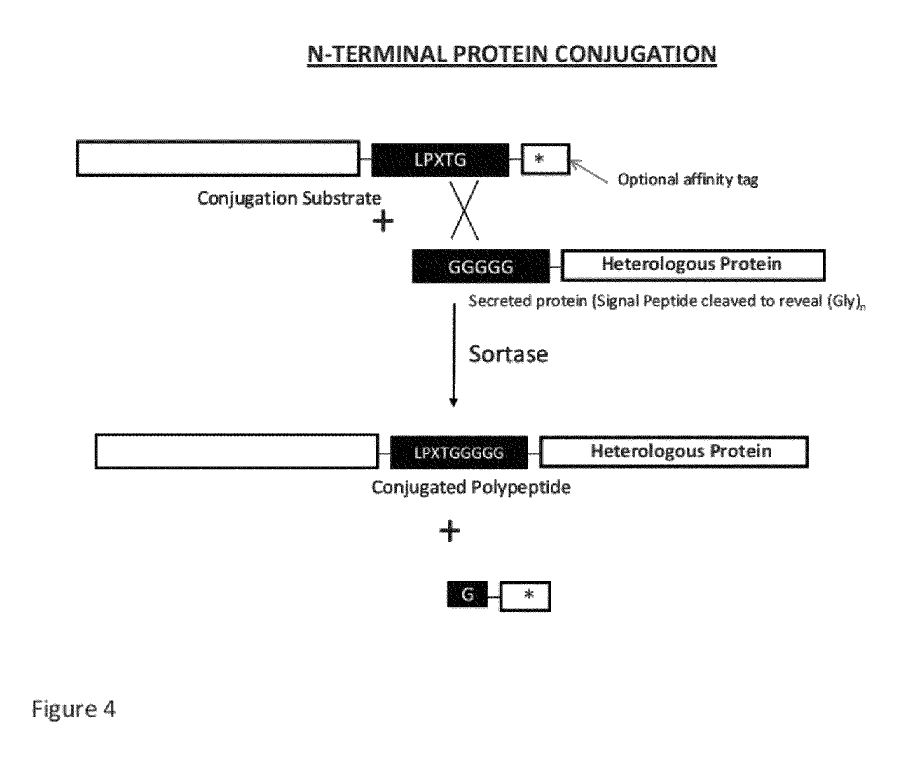

FIG. 5 depicts an exemplary procedure for producing a PEGylated Interferon molecule. First sequence is (SEQ ID NO:15), second sequence is (SEQ ID NO:20), third sequences LPXTG (SEQ ID NO:6)-tagged mPEG, fourth sequence is (SEQ ID NO:21)

### DETAILED DESCRIPTION OF THE INVENTION

Methods are provided herein for expressing a heterologous polypeptide in cell culture under conditions which allow the heterologous polypeptide to be conjugated to a molecule of interest without the need for significant additional processing steps relative to standard cell culture protocols. The methods involve expressing the heterologous polypeptide in the presence of a cell surface-associated bacterial sortase with a sortase catalytic domain exposed to the culture medium. The sortase is capable of specifically ligating the heterologous polypeptide to a molecule of interest added to the culture medium. Advantageously, the methods provide a simple, cost-effective approach for conjugating a heterologous polypeptide to any molecule of interest using established materials and protocols.

A “polypeptide” or “protein” refers to a molecule comprising at least two covalently attached amino acids. A polypeptide can be made up of naturally occurring amino acids and peptide bonds and/or synthetic peptidomimetic residues and/or bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs are compounds that have the same basic chemical structure as a naturally occurring amino acid, i.e., an a carbon bound to hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

As used herein, the term “heterologous polypeptide” refers to a polypeptide encoded by a DNA molecule that does not exist naturally within a given host cell. DNA molecules comprising DNA that is endogenous to the host cell species are considered to be non-naturally occurring, and to thus encode heterologous proteins, so long as the host cell DNA is combined with non-host cell DNA. For example, a polypeptide encoded by a non-host cell DNA segment linked to a host cell promoter is considered to be a heterologous polypeptide. Similarly, a polypeptide encoded by an endogenous gene operably linked with a promoter derived from a non-host cell gene is also considered to be a heterologous polypeptide.

The term “expression,” as used herein, refers to the biosynthesis of a gene product. For example, in the case of a structural gene, expression involves transcription of the structural gene into mRNA and the translation of mRNA into one or more polypeptides.

An “isolated” polypeptide is substantially free of materials other than those comprising the polypeptide in its active form, including, e.g., other proteins and materials derived from the host cells in which the polypeptide is produced, culture medium, growth factors, and the like. In some embodiments, an isolated polypeptide has less than about 30%, or less than about 20%, or less than about 10%, or less than about 5% (by dry weight) of contaminating materials.

A “molecule of interest” to be conjugated (ligated) to a heterologous polypeptide according to methods provided herein can be any molecule suitable for conjugation to a polypeptide. The molecule of interest can confer any of a number of possible functionalities to the heterologous polypeptide, such as but not limited to, altered physico-chemical properties, such as solubility and/or stability; altered pharmacokinetic properties, such as bioavailability, clearance rate, and/or plasma half-life; and/or altered biological activity, such as immunogenicity and/or antigenicity. In one embodiment, the molecule of interest comprises protein, nucleic acid, carbohydrate, lipid, and/or fatty acid etc. In one embodiment, the molecule of interest is a pharmacological carrier molecule, a reporter molecule (e.g., a reporter enzyme, a fluorescent molecule, a radiolabel, an affinity label, or the like), a small molecule, a peptide, a lipid, a carbohydrate, an affinity tag (e.g. His₆), or the like.

A “host cell,” as used herein, is any cell capable of being grown and maintained in cell culture under conditions allowing for production and recovery of useful quantities of a heterologous polypeptide. Host cells can be unmodified cells or cell lines, or cell lines which have been genetically modified (e.g., to facilitate production of heterologous polypeptides). In one embodiment, the host cell is a eukaryotic host cell. Eukaryotic host cells are generally preferred for the production of heterologous polypeptides that are intended for use as biotherapeutic agents or are otherwise intended for administration to or consumption by humans. For example, a eukaryotic host cell is generally preferred for production of heterologous polypeptides requiring post-translational modification (e.g., glycoproteins) and/or folding of multiple polypeptide chains (e.g., antibodies) for optimal biological activity.

As used herein, the term “sortase” refers to a polypeptide having a catalytic domain with activity capable of i) selectively cleaving a backbone amide bond of a polypeptide (peptidase activity) at a “sortase recognition sequence,” and ii) selectively catalyzing the formation of an amide bond between the terminal carboxyl group created by the cleavage and the free primary amino (NH₂—CH₂—) group of a “polyglycine” sequence (transamidase activity). Sortases are typically derived from enzymes expressed on the surface of Gram-positive bacteria which cleave cell surface proteins and link them to cell wall proteoglycans.

The term “polyglycine” as used with respect to a sortase ligation sequence refers to a (Gly)ₙ sequence, wherein n is between 1 and about 10, or more preferably between 2 and about 5, and even more preferably 2 or 3, glycine residues. An “N-terminal” polyglycine sequence is located at the N-terminus of a polypeptide, such that the polypeptide comprises a free primary amino (NH₂—CH₂—) group at its N-terminus An “N-terminal” polyglycine sequence can also include an internal polyglycine sequence that is capable of forming a polyglycine sequence under applicable conditions, e.g., by cleavage of an N-terminal peptide sequence by an endogenous host cell enzyme, or by specific proteolytic cleavage in vitro.

A “soluble sortase” is a catalytically active sortase fragment comprising a sortase catalytic domain without the native hydrophobic, membrane anchoring, transmembrane domain with which it is normally associated, such that the sortase is generally soluble in aqueous environments. In some preferred embodiments, the soluble sortases provided herein are expressed in cultured host cells so that the sortase catalytic domain is accessible to and soluble within the extracellular culture medium. Soluble sortases have been produced in the art, for examplesee H. Ton-Tat et al. Proc. Natl. Acad Sci. USA 1999, 96 12424-12429; and U. llangovan et al. Proc. Natl. Acad Sci. USA 2001, 98, 6056-6061.

The term “sortase ligation sequence” refers to an amino acid sequence that is capable of being selectively ligated to a second amino acid sequence by a sortase. A sortase ligation sequence can be either a sortase recognition sequence or a polyglycine sequence. A “complementary sortase ligation sequence” refers to a second sortase ligation sequence that is capable of being selectively ligated to a first sortase ligation sequence. For example, if the first sortase ligation sequence is a sortase recognition sequence, the complementary sortase ligation sequence is a polyglycine sequence, and vice versa. Similarly, if the first sortase ligation sequence is referred to generically, the complementary sortase ligation sequence is generically complementary to the first sortase ligation sequence.

As used herein, the term “conjugation substrate” refers to a molecule of interest to be conjugated to a heterologous polypeptide linked to a sortase ligation sequence. The conjugation substrate typically comprises a sortase ligation sequence that is complementary to the sortase ligation sequence associated with the heterologous polypeptide. In some embodiments, the conjugation substrate is of the structure S-L-R, wherein S is a sortase ligation sequence, L is an optional linker and R is any molecule of interest, such as but not limited to, a pharmacological carrier molecule, a reporter molecule (e.g., a reporter enzyme, a fluorescent molecule, a radiolabel, an affinity label, or the like), a small molecule, a peptide, a lipid, a carbohydrate, an affinity tag, or the like. In some embodiments, L comprises a spacer polypeptide.

As used herein, the term “signal sequence” or “signal peptide” denotes a peptide sequence, or a DNA sequence that encodes a peptide sequence, that when present within a larger polypeptide targets the polypeptide for secretion by the cell in which it is synthesized. Signal peptides are often cleaved from the larger polypeptide by endogenous enzymes during transit through the secretory pathway of the host cell. A “eukaryotic signal sequence” is a signal peptide, or a DNA sequence that encodes a signal peptide, which is capable of targeting a polypeptide for secretion by a eukaryotic host cell.

As used herein, the term “transmembrane domain” refers to a hydrophobic amino acid sequence that targets and anchors a translated polypeptide comprising the transmembrane domain to the plasma membrane of a host cell. A “type I transmembrane domain” refers to a transmembrane domain which is capable of anchoring a translated polypeptide comprising the transmembrane domain to the plasma membrane of a host cell in a type I orientation. As used herein, the term “type I orientation” refers to an orientation in which the C-terminal portion of the protein resides within the membrane and/or the cytoplasm and the N-terminal portion of the protein is exposed to the cell surface. A “type II transmembrane domain” refers to a transmembrane domain which is capable of anchoring a translated polypeptide comprising the transmembrane domain to the plasma membrane of a host cell in a type II orientation. As used herein, the term “type II orientation” refers to an orientation of a membrane protein in which the N-terminal portion of the protein resides within the membrane and/or the cytoplasm and the C-terminal portion of the protein is exposed to the cell surface.

A nucleotide or amino acid sequence is “operably linked” to another nucleotide or amino acid sequence when it is placed into a functional relationship in relation to the other sequence. For example, an amino acid sequence comprising a secretory signal peptide is operably linked to an amino acid sequence comprising a heterologous polypeptide where the signal peptide is capable of directing secretion of the heterologous polypeptide upon expression of the signal peptide and the heterologous polypeptide in a host cell. As a further example, a promoter or enhancer nucleotide sequence is operably linked to a coding nucleotide sequence if the promoter or enhancer is capable of affecting the transcription of the coding sequence in a host cell. Similarly, a ribosome binding site nucleotide sequence is operably linked to a coding nucleotide sequence if the ribosome binding site is capable of facilitating translation of the corresponding primary transcript in a host cell. In some embodiments, operably linked nucleotide sequences are contiguous and in the same reading frame, whereas in other embodiments operably linked sequences may be non-contiguous and/or in different reading frames.

In some embodiments, two or more operably linked amino acid sequences comprise a fusion protein. As used herein, the term “fusion protein” refers to a hybrid protein encoded by nucleotide sequences derived from two or more genes such that the fusion protein comprises as least two amino acid sequences that are not associated with each other in nature. For example, a fusion protein might comprise a eukaryotic signal sequence suitable for targeting the protein for secretion in a eukaryotic host cell and a soluble sortase normally expressed only in bacteria.

As used herein, the term “affinity tag” denotes a polypeptide segment that is capable of conferring certain binding properties to a larger polypeptide of which it is part. For example, in some embodiments, an affinity tag confers selective binding of a polypeptide to a second polypeptide or other moiety, allowing for purification, substrate attachment, detection, and the like of the polypeptide

As used herein, the term “spacer” refers to a polypeptide sequence which provides physical separation and/or flexibility between two or more portions of a polypeptide.

A “linker” refers to any chemical moiety capable of functionally linking two or more groups, such as a sortase ligation sequence and a molecule of interest. In some embodiments, a linker may comprise a spacer peptide.

The terms “N-terminal to” and “C-terminal to” are used herein to denote the position of a structural feature of a polypeptide relative to other structural features within the same polypeptide chain. A feature is “N-terminal” to another if it is closer to the amino-terminal end of the polypeptide, and a feature is “C-terminal” to another if it is closer to the carboxy-terminal end of the polypeptide.

“Contacting” a cell with a conjugation substrate according to methods provided herein refers to the addition of the conjugation substrate to the culture medium in a manner that allows the cell surface-associated sortase to ligate the conjugation substrate to the heterologous polypeptide. In some embodiments, the contacting includes culturing the cells for a defined period of time in the presence of the conjugation substrate. In other embodiments, the contacting includes culturing the cells for a variable period of time until a desired endpoint or other indicator is achieved.

In one aspect, methods are provided herein for producing a conjugated polypeptide, comprising:

expressing a first nucleotide sequence encoding a first fusion protein in a cultured host cell, the first fusion protein comprising a first eukaryotic signal sequence, a transmembrane domain and a soluble sortase, wherein the first signal sequence targets the first fusion protein for secretion by the host cell and the transmembrane domain anchors the first fusion protein in the plasma membrane of the cell with the sortase exposed to the extracellular medium;

expressing a second nucleotide sequence encoding a second fusion protein in a cultured host cell, the second fusion protein comprising a second eukaryotic signal sequence, a heterologous polypeptide, and a first sortase ligation sequence, wherein the second eukaryotic signal sequence targets the second fusion protein for secretion by the host cell;

contacting the cell with a conjugation substrate comprising a second sortase ligation sequence operably linked to a molecule of interest, wherein one of the first or second sortase ligation sequences comprises a sortase recognition sequence and the other of the first or second sortase ligation sequences comprises a polyglycine sequence;

maintaining the cell under conditions which allow the sortase to cleave the sortase recognition sequence and ligate the complementary sortase ligation sequence to the cleaved sortase recognition sequence to form a conjugated polypeptide; and

isolating the conjugated polypeptide. Conditions which allow the sortase to cleave the sortase recognition sequence and ligate the complimentary sortase ligation sequence (e.g. allow conjugation of substrate), include for example, standard cell growth conditions known to those of skill in the art, e.g. for mammalian cells; 37° C., 5% CO₂, and an appropriate cell culture medium. The cell culture medium may vary depending upon the host cell and can be determined readily by those of skill in the art.

In one embodiment, the methods comprise:

expressing a first nucleotide sequence encoding a first fusion protein in a cultured host cell, the first fusion protein comprising a first eukaryotic signal sequence, a transmembrane domain and a soluble sortase, wherein the first signal sequence targets the first fusion protein for secretion by the host cell and the transmembrane domain anchors the first fusion protein in the plasma membrane of the cell with the sortase exposed to the extracellular medium;

expressing a second nucleotide sequence encoding a second fusion protein in the host cell, the second fusion protein comprising a second eukaryotic signal sequence, a heterologous polypeptide, and a first sortase ligation sequence, wherein the second eukaryotic signal sequence targets the second fusion protein for secretion by the host cell;

contacting the cell with a conjugation substrate comprising a second sortase ligation sequence operably linked to a molecule of interest, wherein one of the first or second sortase ligation sequences comprises a sortase recognition sequence and the other of the first or second sortase ligation sequences comprises a polyglycine sequence;

maintaining the cell under conditions which allow the sortase to cleave the sortase recognition sequence and ligate the complementary sortase ligation sequence to the cleaved sortase recognition sequence to form a conjugated polypeptide; and

isolating the conjugated polypeptide.

In another embodiment, the methods comprise:

expressing a nucleotide sequence encoding a fusion protein in a cultured host cell, the fusion protein comprising a eukaryotic signal sequence, a heterologous polypeptide, and a first sortase ligation sequence, wherein the eukaryotic signal sequence targets the fusion protein for secretion by the host cell;

culturing the host cell in the presence of a second cell modified to express a sortase on the cell surface with a sortase catalytic domain exposed to the culture medium;

contacting the second cell with a conjugation substrate comprising a second sortase ligation sequence operably linked to a molecule of interest, wherein one of the first or second sortase ligation sequences comprises a sortase recognition sequence and the other of the first or second sortase ligation sequences comprises a polyglycine sequence;

maintaining the host cell under conditions which allow the sortase to cleave the sortase recognition sequence and ligate the complementary sortase ligation sequence to the cleaved sortase recognition sequence to form a conjugated polypeptide; and

isolating the conjugated polypeptide.

In some embodiments, the sortase is sortase A (SrtA) or a catalytically active fragment, derivative, or variant thereof. For example, in some preferred embodiments, the sortase is sortase A of Staphylococcus aureus (Sa-SrtA) or a catalytically active fragment, derivative, or variant thereof. In some embodiments, the sortase is a soluble fragment of sortase A comprising the C-terminal catalytic domain (e.g., from about amino acid 60 to about amino acid 206 of Sa-SrtA). The nucleotide sequence Sa-SrtA gene (SEQ ID NO:1) and the amino acid sequence of the encoded protein (SEQ ID NO:2) as well as methods for cloning, expressing, isolating, and assaying the activity of Sa-SrtA are known in the art and are disclosed, e.g., in U.S. Pat. Nos. 6,773,706 and 7,101,692, which are incorporated by reference herein.

Sortase A typically comprises a hydrophobic N-terminal domain (e.g., residues 1 to about 25 of Sa-SrtA) which functions as both a signal peptide and a membrane anchoring domain, a central linker domain (e.g., from about residue 26 to about residue 59 of Sa-SrtA), and a C-terminal catalytic domain (e.g., from about residue 60 to about residue 206 of Sa-SrtA). The hydrophobic N-terminal domain anchors endogenous sortase A enzymes within the bacterial cell wall in a type II orientation.

As used herein, “sortase A catalytic activity” refers to the ability of a sortase to catalyze the cleavage of a polypeptide within a sortase A consensus recognition sequence and ligate the free primary amino group (NH₂—CH₂—) of a polyglycine sequence to the free C-terminal carboxyl group of the cleaved polypeptide. Sortase A catalytic activity can be assayed using methods known in the art, including those described in the Examples herein. The crystal structure of SrtA complexed with a substrate has been determined allowing catalytic active domains of sortase A proteins from various Gram-positive bacterium to be easily discerned by those of skill in the art, see for example, Y. Zong et al. J. Biol Chem. 2004, 279, 31383-31389, which is incorperated herein by reference.

In some embodiments, the sortase is sortase B (SrtB) or a catalytically active fragment, derivative, or variant thereof. For example, in some embodiments, the sortase is sortase B of Staphylococcus aureus (Sa-SrtB) or a catalytically active fragment, derivative, or variant thereof. In some embodiments, the sortase is a soluble fragment of sortase B comprising the central catalytic domain (e.g., from about amino acid 30 to about amino acid 229 of Sa-SrtB). The nucleotide sequence Sa-SrtB gene (SEQ ID NO:3) and the amino acid sequence of the encoded protein (SEQ ID NO:4) as well as methods for cloning, expressing, isolating, and assaying the activity of Sa-SrtB are known in the art and are disclosed, e.g., in U.S. Pat. Nos. 6,773,706 and 7,101,692, which are incorporated by reference herein.

Native sortase B enzymes typically comprise an N-terminal signal peptide (e.g., residues 1 to about 29 of Sa-SrtB), a catalytic domain located C-terminal to the signal peptide (e.g., from about residue 30 to about residue 229 of Sa-SrtB), and a C-terminal hydrophobic domain which functions as a membrane anchoring domain (e.g., from about residue 230 to about residue 244 of Sa-SrtB). The hydrophobic C-terminal domain anchors endogenous sortase B enzymes within the bacterial cell wall in a type I orientation.

As used herein, the term “sortase B catalytic activity” refers to the ability of a sortase to catalyze cleavage of a polypeptide within a sortase B consensus recognition sequences and ligate the free primary amino group (NH₂—CH₂—) of a polyglycine sequence to the free C-terminal carboxyl group of the cleaved polypeptide. The crystal structure of SrtB has been determined, thus catalytic active domains of sortase B proteins from various Gram-positive bacterium can be discerned by those of skill in the art, see for example, R. Zhang et al. Structure, Volume 12, Issue 7, 1147-1156, 1 Jul. 2004; and Y. Zong et al. Structure, Volume 12, 105-112, 2004, which are incorperated herein by reference. Sortase B catalytic activity can be assayed using methods known in the art

In further embodiments, the sortase is derived from a Gram-positive bacterium other than Staphylococcus aureus. For example, in some embodiments, the sortase is SrtA from a species selected from: Bacillus anthracis (e.g. NCBI Reference Sequence: ZP_{—}00391074.1, GI:65318115), Bacillus cereus (e.g. NCBI Reference Sequence: ZP_{—}04310252.1, GI:229183020), Bacillus halodurans (e.g. GeneBank BAB07729.1, GI:10176635), Clostridium acetobutylicum (e.g. NCBI Reference Sequence: NP_{—}346846.1, GI:15893497, SortaseD), Clostridium perfringens (e.g. GeneBank: EDT72453.1, GI:177910051), Clostridium tetani (e.g. GeneBank: AAO35768.1 GI:28203326, SortaseD), Enterococcus faecalis (e.g. NCBI Reference Sequence: ZP_{—}05594184.1, GI:257417190), Lactobacillus plantarum (e.g. NCBI Reference Sequence: YP_{—}003923735.1, GI:308179607), Lactococcus lactis (e.g. GeneBank: ADA64843.1, GI:281375330), Listeria innocua (e.g. NCBI Reference Sequence: NP_{—}470268.1, GI:16800000), Listeria monocytogenes (e.g. NCBI Reference Sequence: YP_{—}002757655.1, GI:226223548), Stephylococcus epidermis (e.g. NCBI Reference Sequence: NP_{—}765035.1, GI:27468398), Streptococcus agalactiae (e.g. NCBI Reference Sequence: NP_{—}687973.1, GI:22537122), Streptococcus gordonii (e.g. GeneBank: BAC66116.1 GI:29134847), Streptococcus mutans (e.g. GeneBank: BAC78819.1 GI:32400378), Streptococcus phenumoniae (e.g. NCBI Reference Sequence: YP_{—}003876834.1, GI:307067868), Streptococcus pyogenes (e.g. GeneBank: ACI61212.1 GI:209540636), and Streptococcus suis (e.g.GeneBank: ABY47175.1 GI:163866429).

In further embodiments, the sortase is SrtB from a species selected from: Bacillus anthracis (e.g. NCBI Reference Sequence: ZP_{—}05199373.1 GI:254741686), Bacillus cereus (e.g. GenBank: ACK61945.1 GI:218161953), Bacillus halodurans (GeneBank: BAB07013.1, GI:10175917), Clostridium perfringens (e.g. GenBank: ABG84849.1, GI:110675862), Listeria innocua (e.g. GenBank: CAC97513.1, GI:16414797), and Listeria monocytogenes (e.g. NCBI Reference Sequence: ZP_{—}07074006.1, GI:300764010).

In some preferred embodiments, the sortase is selected according to the degree of sequence homology with Sa-SrtA or Sa-SrtB. Sortases having a desired degree of homology to Sa-SrtA or Sa-SrtB can be identified by, e.g., using the Sa-SrtA and/or Sa-SrtB nucleotide sequences as query sequences in a search against public databases to identify related sequences. For example, in one embodiment the sortase comprises an amino acid sequence homologus to amino acids 60-206 of Sortase A of S. aureus (SEQ ID NO:2), e.g. an amino acid sequence that is at least 55%, at least 60%, at least 65%, at least 70%, at least 75%, at least 80%, at least 85%, at least 90%, or at least 95% or higher, homologous thereto. In one embodiment, the sortase comprises an amino acid sequence homologous to amino acids 30-229 of Sortase B of S. aureus (SEQ ID NO:4), e.g. an amino acid sequence that is at least 55%, at least 60%, at least 65%, at least 70%, at least 75%, at least 80%, at least 85%, at least 90%, at least 95% or higher, homologous thereto.

One test for comparing two nucleic acids is to determine the percentage of identical nucleotide sequences shared between the nucleic acids. The term “% identity,” in the context of two or more nucleic acids or polypeptide sequences, refers to two or more sequences or subsequences that have a specified percentage of amino acid residues or nucleotides that are the same (e.g., about 10%, or more preferably 15%, 20%, 25%, 30%, 31%, 32%, 33%, 34%, 35%, 36%, 37%, 38%, 39%, 40%, or higher identity over a specified region). Percent identity is typically determined by comparing sequences that have been aligned for maximum correspondence over a comparison window or other designated region.

A “comparison window,” as used herein, refers to a segment of any number of contiguous amino acid or nucleic acid residues within one or more optimally aligned sequences to be compared. Methods for aligning sequences for comparison are well-known in the art. For example, alignment of sequences for comparison can be conducted by the local homology algorithm of Smith & Waterman, Adv. Appl. Math., 1981, 2:482, the homology alignment algorithm of Needleman & Wunsch, J. Mol. Biol., 1970, 48:443, the search for similarity method of Pearson & Lipman, Proc. Nat'l. Acad. Sci. USA, 1988, 85:2444, or the computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection.

Examples of preferred algorithms for determining percent sequence identity and sequence similarity are the BLAST and BLAST 2.0 algorithms, which are described in Altschul et al., Nuc. Acids Res., 25: 3389-3402, 1977 and Altschul et al., J. Mol. Biol. 215: 403-410, 1990, respectively. BLAST and BLAST 2.0 can be used, with the parameters described herein, to determine percent sequence identity of nucleic acids and proteins described herein. Software for performing BLAST analyses is publicly available through the National Center for Biotechnology Information (http://www.ncbi.nlm.nih.gov/).

In some embodiments, the sortase has at least 25%, or preferably at least 30%, or more preferably at least 35% or more identity with the nucleic acid sequence of Sa-SrtA or Sa-SrtB. In further embodiments, the sortase has at least 35%, or preferably at least 40%, or more preferably at least 45% similarity with the amino acid sequence of Sa-SrtA or Sa-SrtB.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and are described, e.g., in Current Protocols in Molecular Biology, John Wiley & Sons, NY, (1989). Aqueous and non-aqueous methods are described therein and either can be used. An example of stringent conditions includes hybridization in 6× sodium chloride/sodium citrate (SSC) at about 45° C., followed by one or more washes in 0.2× SSC, 0.1% SDS at 50° C. Another example of stringent conditions includes hybridization in 6× sodium chloride/sodium citrate (SSC) at about 45° C., followed by one or more washes in 0.2×SSC, 0.1% SDS at 55° C. A further example of stringent conditions includes hybridization in 6×sodium chloride/sodium citrate (SSC) at about 45° C., followed by one or more washes in 0.2×SSC, 0. 1% SDS at 60° C. Stringent conditions frequently involve hybridization in 6×sodium chloride/sodium citrate (SSC) at about 45° C., followed by one or more washes in 0.2×SSC, 0.1% SDS at 65° C. Also, stringent conditions can include hybridization in 0.5M sodium phosphate, 7% SDS at 65° C., followed by one or more washes at 0.2×SSC, 1% SDS at 65° C.

Thus, in some embodiments, the sortase is encoded by a nucleic acid capable of specifically hybridizing to a nucleic acid encoding SrtA or SrtB of Staphylococcus aureus under stringent conditions.

In some embodiments, the sortase is a variant of SrtA or SrtB of Staphylococcus aureus or another Gram-positive bacterium having one or more as substitutions, deletions, insertions, and/or other modifications relative to the native nucleotide and/or amino acid sequence. In some embodiments, the variant comprises one or more conservative amino acid substitutions relative to SrtA or SrtB of Staphylococcus aureus or another Gram-positive bacterium. In further embodiments, the variant comprises one or more as amino acid substitutions relative to SrtA or SrtB of Staphylococcus aureus or another Gram-positive bacterium, wherein the one or more as amino acid substitutions are predominantly, e.g., at least 50%, or preferably at least 60%, or more preferably at least 70% or more, conservative substitutions.

“Conservatively modified variants” include variants of both amino acid and nucleic acid sequences. With respect to a particular nucleic acid sequence, a conservatively modified variant refers to those nucleic acids which encode identical or essentially identical amino acid sequences, or if the nucleic acid does not encode an amino acid sequence, to essentially identical sequences. Because of the degeneracy of the genetic code, a large number of functionally identical nucleic acids encode any given protein. Thus, many nucleic acid sequence variations do not alter the sequence of an encoded polypeptide. Such nucleic acid variations are “silent variations.” Nucleic acid sequences disclosed herein which encode a polypeptide also include all possible silent variants of the nucleic acid.

With respect to amino acid sequences, substitutions, deletions or additions which alter, add or delete an amino acid with a chemically similar amino acid are “conservative modifications.” Conservative substitution tables providing functionally similar amino acids are well known in the art. For example, the following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins, 1984).

In some embodiments, the sortase is a variant of Sa-SrtA comprising an amino acid substitution at Trp 194. For example, in some embodiments, the sortase is a W194A Sa-SrtA variant.

In some embodiments, the first sortase ligation sequence comprises a sortase recognition sequence and the second sortase ligation sequence comprises a polyglycine sequence. In such embodiments, the first sortase ligation sequence is preferably located C-terminal of the heterologous polypeptide and/or the second eukaryotic signal sequence.

In other embodiments, the first sortase ligation sequence comprises a polyglycine sequence and the second sortase ligation sequence comprises a sortase recognition sequence. In such embodiments, the second sortase ligation sequence is preferably located C-terminal of the molecule of interest. In further such embodiments, the first sortase ligation sequence is located C-terminal of the second eukaryotic signal sequence and the second eukaryotic signal sequence is capable of being cleaved by a host cell enzyme to generate a polyglycine sequence.

In some embodiments, the sortase recognition sequence comprises a sortase A recognition sequence having the consensus sequence: X₁PX₂X₃G (SEQ ID NO:5), wherein X₁ is Leu, Ile, Val or Met, P is Pro, X₂ is any amino acid, X₃ is Ser, Thr or Ala, and G is Gly, and wherein the sortase cleaves the amide bond between X₃ and G and catalyzes the formation of an amide bond between the C-terminal carboxyl group of X₃ and the NH₂—CH₂— group of the polyglycine sequence. In some embodiments, X₂ is Asp, Glu, Ala, Gln, Lys or Met. In some preferred embodiments, the sortase A recognition sequence is: LPXTG (SEQ ID NO:6), wherein L is Leu, P is Pro, X is any amino acid, T is Thr, and G is Gly.

In some embodiments, the sortase recognition sequence comprises a sortase B recognition sequence having the consensus sequence: NPX₁TX₂ (SEQ ID NO:7), wherein N is Asn, P is Pro, X₁ is Gln or Lys, T is Thr, and X₂ is Asp or Gly, and wherein the sortase cleaves the amide bond between T and X₂ of the recognition sequence and catalyzes the formation of an amide bond between the C-terminal carboxyl group of T and the NH₂—CH₂— group of the polyglycine sequence. In some embodiments, the sortase B recognition sequence is: NPQTN (SEQ ID NO:8), wherein N is Asn, P is Pro, Q is Gln, T is Thr, and G is Gly.

In some preferred embodiments, the polyglycine sequence comprises 1, 2, 3, 4 or 5 consecutively linked glycine residues. In further embodiments, the polyglycine sequence comprises 1, 2, or 3 glycine residues.

Sortase ligation sequences can be incorporated or added to the second fusion protein and/or the conjugation substrate using methods known in the art. Where the heterologous polypeptide and/or molecule of interest comprises one or more sortase ligation sequences as part of their native structure, such native sortase ligation sequences can be removed by, e.g., expressing a variant of the polypeptide and/or molecule of interest without the sortase ligation sequence(s) or by chemically modifying (e.g., blocking) some and/or all of the amino acids comprising the unintended sortase ligation sequence(s).

The first and eukaryotic signal sequence and the transmembrane domain of the first fusion protein and the second eukaryotic signal sequence of the second fusion protein are generally peptide sequences which are capable of targeting a nascent polypeptide for intracellular transport and/or secretion in a eukaryotic host cell. In eukaryotic cells, most secreted and membrane-bound proteins are translocated across the endoplasmic reticulum (ER) membrane concurrently with translation. A signal sequence is generally an N-terminal peptide comprising about 10-20 hydrophobic amino acids which targets the nascent protein from the ribosome to the endoplasmic reticulum (ER) and/or one or more other membrane bound compartments of the secretory pathway, such as the Golgi apparatus and/or lysosomes. Proteins targeted to a compartment of the secretory pathway may remain in one of the secretory organelles or they may proceed through the secretory pathway, at which point they are either secreted into the extracellular space or retained in the plasma membrane.

In some embodiments, the first eukaryotic signal sequence comprises a type I signal sequence typically found in Type I membrane proteins. Type I signal sequences are cleaved by a signal peptidase in the lumen of the ER and the remainder of the protein is secreted from the cell and anchored in the plasma membrane by a separate transmembrane domain. Proteins comprising a transmembrane domain are typically anchored in the plasma membrane in a type I orientation, with the C-terminal end located in the cytosol of the cell and the N-terminal end displayed on the surface of the cell.

In other embodiments, the first eukaryotic signal sequence comprises a “signal anchor sequence” which directs the associated protein to the secretory pathway and also anchors the protein in the plasma membrane. Proteins comprising a signal anchor sequence are typically anchored in the plasma membrane in a type II orientation, in which the N-terminal end is located in the cytosol of the cell and the C-terminal end is displayed on the surface of the cell. Thus, when the first eukaryotic signal sequence comprises a signal anchor sequence, it also serves as the transmembrane domain.

The second eukaryotic signal sequence preferably comprises a type I signal sequence, such that the second eukaryotic signal sequence is removed in the ER prior to secretion of the second fusion protein.

Systems for expressing heterologous proteins as fusion proteins with a signal peptide suitable for secretion and/or cell surface display of the heterologous protein are known in the art, and are described, e.g., in Mottershead et al., Biochem. Biophys. Res. Commun., 238:717 (1997); Yang, U.S. Pat. No. 5,665,590; Steven et al. BioEssays Volume 12, Issue 10, pages 479-484, October 1990, and Lok, U.S. Pat. No. 7,125,973.

For example, secretory signal sequences suitable for use in yeast host cells include the α-factor signal peptide (cf. U.S. Pat. No. 4,870,008), the signal peptide of mouse salivary amylase (Hagenbuchle et al., Nature, 289: 643-646 (1981)), modified carboxypeptidase signal peptides (Valls et al., Cell, 48: 887-897 (1987)), the yeast BAR1 signal peptide (PCT Pub. No. WO 87/02670), and the yeast aspartic protease 3 (YAP3) signal peptide (cf. M. Egel-Mitani et al., Yeast 6, 1990, pp. 127-137).

Additional exemplary signal sequences are described in Izard et al., Mol Microbiol., 13(5): 765-73 (1994); Bolhuis et al., Microbiol Mol Biol Rev., 64 (3): 515-47 (2000); and Giga-Hama et al., Biotechnol. Appl. Biochem., 30: 235-244 (1999), each of which is herein incorporated by reference.

In some embodiments, a signal sequence is capable of being selectively cleaved from an expressed fusion protein by an endogenous enzyme of the host cell. Cleavage of the signal peptide can occur before, after, or concurrently with secretion of the fusion protein into the extracellular medium.

In some embodiments, a sequence encoding a leader peptide is inserted downstream of the signal sequence and upstream of the DNA sequence encoding the coding sequence. The leader peptide directs expressed polypeptides operably linked to the leader peptide from the endoplasmic reticulum to the Golgi apparatus and further to a secretory vesicle for secretion into the culture medium. An exemplary leader peptide is the yeast alpha-factor leader (described, e.g., in U.S. Pat. No. 4,546,082, U.S. Pat. No. 4,870,008, EP 16 201, EP 123 294, EP 123 544 and EP 163 529). Alternatively, the leader peptide may be a synthetic leader peptide, such as those described in PCT Pub. Nos. WO 89/02463 and WO 92/11378.

A transmembrane domain can comprise any peptide which is capable of targeting and anchoring a translated polypeptide to the plasma membrane of a host cell. In various embodiments, the transmembrane domain is between about 15 and 35 amino acids in length, or more preferably between about 20 and 31 amino acids in length. A transmembrane domain preferably comprises a membrane spanning region which is capable of assuming a structure (e.g., an alpha helix) which spans the plasma membrane of a host cell under physiological conditions. The membrane spanning region typically comprises at least 50%, or more preferably at least 80% or more hydrophobic amino acid residues, such as Ala, Leu, Val, Ile, Pro, Phe or Met. In some embodiments, the membrane spanning region may be flanked on either or both sides by one or more residues which disrupt the structure of the membrane spanning region (e.g., proline) or which are energetically unstable in the hydrophobic environment of the membrane (e.g., charged residues). The hydrophobic and flanking residues are preferably organized such that non-polar residues are in contact with the membrane interior and charged or polar residues are in contact with the aqueous phase.

In some embodiments, the transmembrane domain is a synthetic peptide. For example, the membrane spanning region of a synthetic transmembrane domain may be designed to assume an alpha helical structure by constructing it from alpha helix-promoting amino acid residues, such as Ala, Asn, Cys, Gln, His, Leu, Met, Phe, Trp, Tyr or Val, or more preferably hydrophobic alpha helix-promoting residues, such as Ala, Met, Phe, Trp or Val.

In some embodiments, the transmembrane domain is derived from a naturally occurring membrane-spanning or cell surface protein. For example, amphipathic alpha-helices that span a lipid membrane bilayer can be identified in primary structures using a secondary structure prediction algorithm which selects segments of an appropriate size (e.g., greater than 15-20 residues) based on sequence similarity to a superfamily of known proteins. For example, the programs “TmPred” and “TopPredII” can predict membrane-spanning regions and their orientation by comparison of sequences to a database of transmembrane proteins present in the SwissProt database (e.g., Gunnar von Heijne, J. Mol. Biol. 225:487-494 (1992); Hoppe-Seyler, Biol. Chem., 347:166 (1993); and Claros, et al., Comput Appl Biosci. 10(6):685-686 (1994)).

In further embodiments, the transmembrane domain comprises a lipid-based membrane anchor, such as a myristyl group, a farnesyl group, a geranyl-geranyl group, a GPI-anchor, or an N-acyl diglyceride group. For example, in some embodiments, the first fusion protein further comprises a C-terminal signal peptide that directs a host cell enzyme to cleave the C-terminal signal peptide and attach a glycosylphosphatidylinositol (GPI) anchor at the C-terminal end of the cleaved protein.

In some embodiments, the sortase is separated from the transmembrane domain by a spacer peptide which reduces steric hindrance between the cell surface and the sortase catalytic domain.

Methods and compositions provided herein can be used to conjugate essentially any heterologous protein to any molecule of interest. Non-limiting examples of heterologous polypeptides that can be produced according to methods provided herein include receptors, membrane proteins, cytokines, chemokines, hormones, enzymes, growth factors, growth factor receptors, antibodies, antibody derivatives and other immune effectors, interleukins, interferons, erythropoietin, integrins, soluble major histocompatibility complex antigens, binding proteins, transcription factors, translation factors, oncoproteins or proto-oncoproteins, muscle proteins, myeloproteins, neuroactive proteins, tumor growth suppressors, structural proteins, and blood proteins (e.g., thrombin, serum albumin, Factor VII, Factor VIII, Factor IX, Factor X, Protein C, von Willebrand factor, etc.). In some embodiments, the heterologous polypeptide is a glycoprotein or other polypeptide which requires post-translational modification, such as deamidation, glycation, or the like, for optimal activity.

Conjugation substrates described herein are generally of the structure S-L-R, wherein S is a sortase ligation sequence (e.g., a sortase recognition sequence or a polyglycine), L is an optional linker and R is any molecule of interest.

The conjugation substrate may comprise any molecule of interest so long as it is capable of being operably linked to a sortase ligation sequence. Non-limiting examples of molecules of interest include: a peptide, a polypeptide, a lipid molecule, a sugar molecule, a nucleic acid, a reporter molecule, a toxin, a therapeutic agent, a nanoparticle, a resin, a cell, a virus particle, an adjuvant molecule, or a polymer. (e.g., a hydrophilic polymer).

In some embodiments, the molecule of interest comprises, consists essentially of, or consists of a member of a prosthetic binding group, such as biotin/avidin, biotin/streptavidin, maltose binding protein/maltose, glutathione S-transferase/glutathione, metal/polyhistidine, antibody/epitope, antibody/antigen, antibody/protein A or protein G, hapten/anti-hapten, folic acid/folate binding protein, vitamin B 12/intrinsic factor, nucleic acid/complementary nucleic acid, sulfhydryl/maleimide, sulfhydryl/haloacetyl derivative, amine/isotriocyanate, amine/succinimidyl ester, or amine/sulfonyl halides.

In some embodiments, the molecule of interest comprises, consists essentially of, or consists of a small molecule, such as but not limited to, a peptide, a peptidomimetic (e.g., a peptoid), an amino acid, an amino acid analog, a polynucleotide or polynucleotide analog, a nucleotide or nucleotide analog, or an organic or inorganic compound having a molecular weight between about 500 and about 10,000.

In some embodiments, the molecule of interest comprises, consists essentially of, or consists of a second polypeptide. The polypeptide can be any polypeptide. For example, a protein which is difficult to produce in a cell (e.g., either due to toxicity), can be expressed as two fragments which can be joined using the methods described herein (e.g., first portion of the protein can be attached to the second portion, reconstituting an active protein using the methods described herein).

In some embodiments, the molecule of interest comprises, consists essentially of, or consists of a reporter molecule, such as a fluorescent molecule (e.g., umbelliferone, fluorescein, fluorescein isothiocyanate, rhodamine, dichlorotriazinylamine fluorescein, dansyl chloride or phycoerythrin); a radioisotope (e.g., Cu-64, Ga67, Ga-68, Zr-89, Ru-97, Tc-99, Rh-105, Pd-109, In-111, I-123, I-125, I-131, Re-186, Re-188, Au-198, Pb-203, At-211, Pb-212 or Bi-212); a detectable enzyme (e.g., horseradish peroxidase, alkaline phosphatase, p-galactosidase, or acetylcholinesterase); a luminescent material (e.g., luminol); or a bioluminescent material (e.g., luciferase, luciferin, or aequorin).

In some embodiments, the molecule of interest comprises, consists essentially of, or consists of a biologically active molecule, such as a toxin (e.g., abrin, ricin A, pseudomonas exotoxin or diphtheria toxin).

In some embodiments, the molecule of interest comprises the heterologous polypeptide itself, such that the heterologous polypeptide is cyclized by the conjugation. For example, in some embodiments, the heterologous polypeptide comprises a first sortase ligation sequence at its N-terminus (e.g., a polyglycine sequence) and a complementary sortase ligation sequence located C-terminal of the first sortase ligation sequence, such that the sortase cyclizes the polypeptide. Advantageously, cyclized proteins often exhibit desired properties relative to the corresponding linear protein, such as enhanced solubility, enhanced stability, enhanced plasma half-life and/or decreasing immunogenicity. In other embodiments, the heterologous polypeptide can be ‘chained’ (e.g., dimerized, trimerized, etc).

In some embodiments, the conjugation substrate comprises a molecule of interest (R) which contains a primary amino (NH₂—CH₂—) group in addition to the sortase ligation sequence. Examples of molecules of interest comprising a primary amino group include, but are not limited to, aminosugars, aminoglycosides, hydroxyamino acids, hydroxyamino acid esters, aminolipids, polyamines, and polypeptides comprising an N-terminal Gly residue.

In some embodiments, the molecule of interest is a water-soluble polymer, non-peptidic polymer with an average molecular weight of about 200 to about 200,000 Daltons, depending on the desired effect on the properties of the heterologous polypeptide. For example, in some embodiments, the molecule of interest comprises, consists essentially of, or consists of a polymeric group, such as polyalkylene oxide (PAO), polyalkylene glycol (PAG), polyethylene glycol (PEG), methoxypolyethylene glycol (mPEG), polypropylene glycol (PPG), branched PEGs, copolymers of ethylene glycol and propylene glycol, polyvinyl alcohol (PVA), polycarboxylate, poly-vinylpyrrolidone, polyethylene-co-maleic acid anhydride, polystyrene-co-maleic acid anhydride, dextran, carboxymethyl-dextran, polyoxyethylated glycerol, polyoxyethylated sorbitol, polyoxyethylated glucose, dextran, polyoxazoline, polyacryloylmorpholine, or a serum protein binding-ligand, such as a compound which binds to albumin (e.g., fatty acids, C₅-C₂₄ fatty acid, aliphatic diacid (e.g. C₅-C₂₄)). Additional polymers useful in methods and compositions provided herein are known in the art and are described, e.g., in U.S. Pat. No. 5,629,384, which is herein incorporated by reference.

When the heterologous polypeptide is a therapeutic protein intended for administration to a mammalian subject, e.g., a human, conjugating a polymer to the protein can confer various beneficial properties to the protein. For example, conjugation of a PEG polymer (PEGylation) is known to significantly improve pharmacokinetic properties of therapeutic proteins, e.g., by increasing effective size, reducing immunogenicity, and/or reducing aggregation. Several PEGylated protein therapeutics are currently on the market or in late-stage clinical testing. For example, PEG-Intron® (PEG-interferon alfa-2b; Schering-Plough) and PEGasys® (PEG-interferon alfa-2a; Roche) are PEGylated variants of interferon alfa (IFNa) which show significantly improved in vivo efficacy relative to the parent proteins in treating hepatitis C.

A wide variety of methods have been described in the art for covalently conjugating PEG and PEG derivatives to active sites of proteins, such as lysine residues or unpaired cysteine residues (e.g., Roberts et al., Adv. Drug Deliv. Rev., 54: 459-476 (2002). In many cases, such methods adversely effect the bioactivity of the PEGylated protein relative to the unmodified protein due to, e.g., attachment at sites affecting the structure and/or activity of the protein, over-modification of the protein (e.g., by attachment at multiple sites), exposure of the protein to harsh coupling conditions, generation of harmful by-products, and/or steric hindrance. Advantageously, methods and compositions provided herein allow for site-specific modification, e.g., PEGylation, of heterologous proteins without significantly reducing specific activity relative to the unmodified proteins.

Thus, in some preferred embodiments, the molecule of interest is a polyethylene glycol (PEG) or derivative thereof PEG is a linear polymer with terminal hydroxyl groups and of the formula HO—CH₂CH₂—(CH₂CH₂O)ₙ—CH₂CH₂—OH, where n is from about 8 to about 4000. In some embodiments, the terminal hydrogen is substituted with a protective group such as an alkyl, alkanol or alkoxy group. For example, a common PEG derivative is methoxy-PEG (mPEG), in which one terminus is a relatively inert methoxy group and the other terminus is a relatively reactive hydroxyl group. Any PEG or PEG derivative can be used in the methods and compositions described herein, including those described, e.g., in U.S. Pat. Nos. 6,515,100, 6,514,491, 6,495,659, 6,448,369, 6,437,025, 6,436,386, 5,932,462, 5,445,090 and 5,900,461, each of which is hereby incorporated by reference.

In some embodiments, the conjugation substrate is a composition of the following structure:

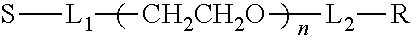

wherein n is 1 to 2500; L₁ and L₂ are independently optional linkers; and S comprises a sortase ligation sequence.

In some embodiments, the conjugation substrate comprises a polymer of the formula:

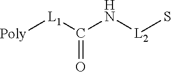

wherein Poly is a water-soluble, non-peptidic polymer with an average molecular weight of about 200 to about 100,000 Daltons; L1 and L2 are independently optional linkers; and S is a sortase ligation sequence. In one embodiment, L1 and L2 are each independently hydrolytically stable linkers. In another embodiment, L1 and L2 are each independently linkers comprising at least 3 contiguous saturated carbon atoms.

In some embodiments, the conjugation substrate comprises a polymer of the formula:

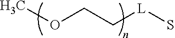

wherein n is 1 to 2,500, L is an optional linker, and S is a sortase ligation sequence.

In some embodiments, the conjugation substrate comprises a polymer of the formula:

In some embodiments, the conjugation substrate is selected from the group consisting of:

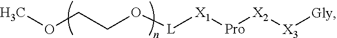

wherein X₁ is Leu, Ile, Val or Met, P is Pro, X₂ is any amino acid, X₃ is Ser, Thr or Ala, and G is Gly;

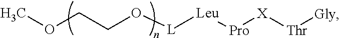

wherein X is any amino acid;

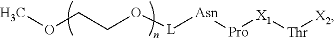

wherein X₁ is Gln or Lys, and X₂ is Asp or Gly.

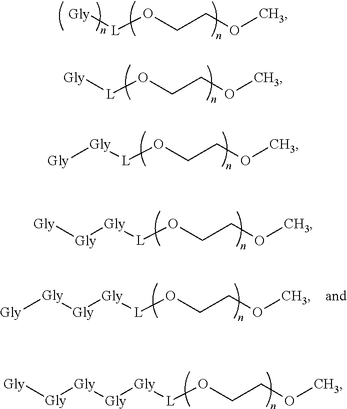

wherein n is 1 to 100,000.

In some embodiments, the composition has a molecular weight of between about 200 and 100,000 daltons, e.g., between about 1,000 and 50,000 Daltons, between about 2,000 and 40,000 Daltons, or between about 5,000 and 25,000 Daltons.

In one embodiment, L is a hydrolytically stable linker. In another embodiment, L is a linker comprising at least 3 contiguous saturated carbon atoms.

In one embodiment, R is a polypeptide having a native sequence comprising one or more consecutive glycine residues at the N-terminus of the polypeptide and S comprises one or more of the N-terminal glycine residues.

In some aspects, the first or second fusion protein and/or the conjugation substrate comprises an affinity tag that can be used to facilitate recovery and/or isolation of the fusion proteins and/or the conjugated polypeptide.

An affinity tag used in a method or composition provided herein can comprise any peptide or other molecule for which an antibody or other specific binding agent is available. Affinity tags known in the art as being useful for protein purification include, but are not limited to, a poly-histidine segment, protein A (e.g., Nilsson et al., EMBO J. 4:1075 (1985); Nilsson et al., Methods Enzymol. 198:3 (1991)), glutathione S transferase (e.g., Smith and Johnson, Gene 67:31 (1988)), Glu-Glu affinity tag (e.g., Grussenmeyer et al., Proc. Natl. Acad. Sci. USA 82:7952 (1985)), substance P, FLAG peptide (e.g., Hopp et al., Biotechnology 6:1204 (1988)), c-myc tags (detected with anti-myc antibodies), calmodulin binding protein, and streptavidin binding peptide.

In some embodiments, an affinity tag described herein allows for selective enrichment of desired conjugation products. In some embodiments, an affinity tag is located N-terminal of a sortase recognition sequence or C-terminal of a polyglycine sequence so that the tag remains associated with the polypeptide after sortase-catalyzed cleavage and ligation. For example, where the affinity tag is operably linked to a fusion protein comprising a heterologous protein and a sortase ligation sequence, the affinity tag is preferably located N-terminal of the sortase ligation sequence (e.g., between the sortase ligation sequence and the heterologous polypeptide or N-terminal of both) if the sortase ligation sequence is a sortase recognition sequence, and C-terminal of the sortase ligation sequence (e.g., between the sortase ligation sequence and the heterologous polypeptide or C-terminal of both) where the sortase ligation sequence is a polyglycine sequence. As such, the affinity tag is retained in the conjugated polypeptide upon cleavage and/or ligation of the sortase recognition sequence by a sortase and affinity purification isolates the intact conjugated polypeptide.

In further embodiments, an affinity tag is located C-terminal of a sortase recognition sequence so that the tag is cleaved from the polypeptide upon sortase-catalyzed cleavage and ligation. For example, where the sortase ligation sequence is a sortase recognition sequence, the affinity tag is located C-terminal of the sortase recognition sequence (i.e., the sortase recognition sequence is between the fusion protein and the affinity tag). Alternatively where the sortase ligation sequence is a polyglycine sequence, the affinity tag is located N-terminal to the polyglycine sequence (i.e., the polyglycine sequence is between the affinity tag and the fusion protein). As such, after performing the cleavage and ligation reactions, the affinity tags are no longer attached to the fusion protein, and fragments containing the affinity tag are easily removed by affinity purification.

In yet further embodiments, the conjugation substrate further comprises a second affinity tag which is different than the affinity tag associated with the heterologous protein such that serially screening for binding to the first and second affinity tags can select for the conjugated polypeptide over the unconjugated conjugation substrate and the unconjugated heterologous polypeptide and/or other non-specific products. Where the conjugation substrate comprises a sortase recognition sequence, the second affinity tag is preferably located N-terminal of the recognition sequence. Where the conjugation substrate comprises a polyglycine sequence, the second affinity tag is preferably located C-terminal of the polyglycine sequence.

In some aspects, the first and/or second fusion protein and/or the conjugation substrate comprises a spacer peptide. For example, in some embodiments, a spacer peptide separates the heterologous polypeptide from a sortase ligation sequence and/or an affinity tag, and/or the sortase ligation sequence from an affinity tag. A spacer peptide can be of any size, e.g., from several to 30 or more amino acid residues, sufficient to serve the intended purpose. Spacer peptides can enhance conformational flexibility between two or more domains of a protein and/or minimize steric interference with the folding and/or function of two or more domains of a protein. A spacer peptide will generally comprise an inert, flexible amino acid sequence, e.g., comprising predominantly glycine, serine, and/or alanine residues. In some embodiments, a spacer peptide sequence can be modified with one or more proline residues at the beginning and/or at the end of the spacer in order to isolate the spacer as a separate functional domain from neighboring domains of the protein. A variety of spacer peptides are known in the art.

The linker (L) of the conjugation substrate can comprise any chemical moiety capable of linking the molecule of interest to the sortase ligation sequence. In some embodiments, L is a spacer peptide. In further embodiments, L can comprise a peptide sequence of about 5 to 9 amino acids, with or without the inclusion of additional groups, such as aliphatic chains of up to 5 carbons in length. In some embodiments, L is labile in that it is capable of being cleaved internally and/or at the site of linkage with the molecule of interest and/or sortase ligation sequence.

A “host cell,” as used herein, is any cell capable of being grown and maintained in cell culture under conditions allowing for production and recovery of useful quantities of a biological product, as defined herein. Host cells can be unmodified cells or cell lines, or cell lines which have been genetically modified (e.g., to facilitate production of a biological product). In some embodiments, the host cell is a cell line that has been modified to allow for growth under desired conditions, such as in serum-free media, in cell suspension culture, or in adherent cell culture.

In some preferred embodiments, the host cell is a mammalian cell. A mammalian host cell may be preferred where the biological product is a recombinant polypeptide, particularly if the polypeptide is a biotherapeutic agent or is otherwise intended for administration to or consumption by humans. In some embodiments, the host cell is a Chinese Hamster Ovary (CHO) cell (ATCC CCL 61), which is a predominant cell line used for the expression of many recombinant proteins. Additional examples of mammalian cells suitable for expressing heterologous polypeptides, including those intended for use as biotherapeutic agents or otherwise intended for administration to humans include, but are not limited to, COS-1 cells (ATCC CRL 1650), baby hamster kidney (BHK) cells (e.g., tk⁻ ts13 BHK cells, Waechter and Baserga, Proc. Natl. Acad. Sci. USA 79: 1106-1110 (1982), incorporated herein by reference; ATCC CRL 10314 and 1632)), Rat Hep I cells (Rat hepatoma; ATCC CRL 1600), Rat Hep II cells (Rat hepatoma; ATCC CRL 1548), TCMK cells (ATCC CCL 139), Human lung cells (ATCC HB 8065), NCTC 1469 cells (ATCC CCL 9.1), DUKX cells (Urlaub and Chasin, Proc. Natl. Acad. Sci. USA 77:4216-4220, 1980) and 293 cells (ATCC CRL 1573; Graham et al., J. Gen. Virol. 36:59-72, 1977).

In some embodiments, the host cell is a CHO cell derivative that has been genetically modified to facilitate production of recombinant proteins or other biological products. For example, various CHO cell strains have been developed which permit stable insertion of recombinant DNA into a specific gene or expression region of the cells, amplification of the inserted DNA, and selection of cells exhibiting high level expression of the recombinant protein. Examples of CHO cell derivatives useful in methods provided herein include, but are not limited to, CHO-K1 cells, CHO-DUKX, CHO-DUKX B1, CHO-DG44 cells, CHO-ICAM-1 cells, and CHO-h1FNγ cells. Methods for expressing recombinant proteins in CHO cells are known in the art and are described, e.g., in U.S. Pat. Nos. 4,816,567 and 5,981,214, herein incorporated by reference in their entirety.

Examples of human cell lines useful in methods provided herein include, but are not limited to, 293T (embryonic kidney), 786-0 (renal), A498 (renal), A549 (alveolar basal epithelial), ACHN (renal), BT-549 (breast), BxPC-3 (pancreatic), CAKI-1 (renal), Capan-1 (pancreatic), CCRF-CEM (leukemia), COLO 205 (colon), DLD-1 (colon), DMS 114 (small cell lung), DU145 (prostate), EKVX (non-small cell lung), HCC-2998 (colon), HCT-15 (colon), HCT-116 (colon), HT29 (colon), HT-1080 (fibrosarcoma), HEK 293 (embryonic kidney), HeLa (cervical carcinoma), HepG2 (hepatocellular carcinoma), HL-60(TB) (leukemia), HOP-62 (non-small cell lung), HOP-92 (non-small cell lung), HS 578T (breast), HT-29 (colon adenocarcinoma), IGR-OV1 (ovarian), IMR32 (neuroblastoma), Jurkat (T lymphocyte), K-562 (leukemia), KM12 (colon), KM20L2 (colon), LAN5 (neuroblastoma), LNCap.FGC (Caucasian prostate adenocarcinoma), LOX IMVI (melanoma), LXFL 529 (non-small cell lung), M14 (melanoma), M19-MEL (melanoma), MALME-3M (melanoma), MCF1OA (mammary epithelial), MCF7 (mammary), MDA-MB-453 (mammary epithelial), MDA-MB-468 (breast), MDA-MB-231 (breast), MDA-N (breast), MOLT-4 (leukemia), NCl/ADR-RES (ovarian), NCI-H226 (non-small cell lung), NCI-H23 (non-small cell lung), NCI-H322M (non-small cell lung), NCI-H460 (non-small cell lung), NCI-H522 (non-small cell lung), OVCAR-3 (ovarian), OVCAR-4 (ovarian), OVCAR-5 (ovarian), OVCAR-8 (ovarian), P388 (leukemia), P388/ADR (leukemia), PC-3 (prostate), PERC6® (E1-transformed embryonal retina), RPMI-7951 (melanoma), RPMI-8226 (leukemia), RXF 393 (renal), RXF-631 (renal), Saos-2 (bone), SF-268 (CNS), SF-295 (CNS), SF-539 (CNS), SHP-77 (small cell lung), SH-SY5Y (neuroblastoma), SK-BR3 (breast), SK-MEL-2 (melanoma), SK-MEL-5 (melanoma), SK-MEL-28 (melanoma), SK-OV-3 (ovarian), SN12K1 (renal), SN12C (renal), SNB-19 (CNS), SNB-75 (CNS) SNB-78 (CNS), SR (leukemia), SW-620 (colon), T-47D (breast), THP-1 (monocyte-derived macrophages), TK-10 (renal), U87 (glioblastoma), U293 (kidney), U251 (CNS), UACC-257 (melanoma), UACC-62 (melanoma), UO-31 (renal), W138 (lung), and XF 498 (CNS).

Examples of rodent cell lines useful in methods provided herein include, but are not limited to, baby hamster kidney (BHK) cells (e.g., BHK21 cells, BHK TK− cells), mouse Sertoli (TM4) cells, buffalo rat liver (BRL 3A) cells, mouse mammary tumor (MMT) cells, rat hepatoma (HTC) cells, mouse myeloma (NS0) cells, murine hybridoma (Sp2/0) cells, mouse thymoma (EL4) cells, Chinese Hamster Ovary (CHO) cells and CHO cell derivatives, murine embryonic (NIH/3T3, 3T3 L1) cells, rat myocardial (H9c2) cells, mouse myoblast (C2C12) cells, and mouse kidney (miMCD-3) cells.

Examples of non-human primate cell lines useful in methods provided herein include, but are not limited to, monkey kidney (CVI-76) cells, African green monkey kidney (VERO-76) cells, green monkey fibroblast (Cos-1) cells, and monkey kidney (CVI) cells transformed by SV40 (Cos-7). Additional mammalian cell lines are known to those of ordinary skill in the art and are catalogued at the American Type Culture Collection catalog (ATCC®, Mamassas, Va.).

In some embodiments, the host cells is suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., hematopoietic cells, lymphoid cells) and cells that have been made suspension-competent by modification or adaptation of attachment-dependent cells (e.g., epithelial cells, fibroblasts).

In some embodiments, the host cell is an attachment dependent cell which is grown and maintained in adherent culture. Examples of human adherent cell lines useful in methods provided herein include, but are not limited to, human neuroblastoma (SH-SY5Y, IMR32 and LAN5) cells, human cervical carcinoma (HeLa) cells, human breast epithelial (MCF1OA) cells, human embryonic kidney (293T) cells, and human breast carcinoma (SK-BR3) cells.

In some embodiments, the host cell is a multipotent stem cell or progenitor cell. Examples of multipotent cells useful in methods provided herein include, but are not limited to, murine embryonic stem (ES-D3) cells, human umbilical vein endothelial (HuVEC) cells, human umbilical artery smooth muscle (HuASMC) cells, human differentiated stem (HKB-Il) cells, and human mesenchymal stem (hMSC) cells.

In some embodiments, the host cell is a plant cell, such as a tobacco plant cell.

In some embodiments, the host cell is a fungal cell, such as a cell from Pichia pastoris, a Rhizopus cell, or an Aspergillus cell.

In some embodiments, the host cell is an insect cell, such as SF9 cells from Spodoptera frugiperda or S2 cells from Drosophila melanogaster.

Conjugation of polypeptides using the methods described herein can be performed directly in the culture in which the cells have grown. For example, in embodiments in which the host cell expresses both the heterologous polypeptide and a cell-surface exposed sortase activity, the host cells secrete the heterologous polypeptide into the medium. To this medium (still containing the host cells), conjugation substrates are added, such that the extracellularly exposed sortase has access to both the heterologous polypeptide and the conjugation substrate. Adjustments can be made to the medium to match conditions ideally suited for sortase activity. For example, the pH (between 7.0-8.0, e.g., between 7.5 and 8.0), ionic strength (˜150 mM NaCl), and concentrations of salts (e.g., 5-10 mM CaCl₂) can be adjusted to provide ideal reaction conditions. Furthermore, compounds present in the culture medium which may be potentially inhibitory for the sortase reaction can be removed, reduced or avoided. In one embodiment, cells can be grown for 24-48 hrs prior to the sortase reaction in a medium reduced in or devoid of primary amines.

The mixture described above containing the cells (with the surface exposed sortase), secreted heterologous polypeptide, and the conjugation substrate are maintained under conditions to allow for the formation of the conjugated polypeptide. In addition to the conditions described above (e.g., pH, CaCl2, etc.), the mixture can be maintained at a defined temperature (e.g., 25° C., 30° C., 33° C., 37° C.). Aliquots of the mixture can be removed over time to monitor the formation of the conjugated polypeptide, the disappearance of the unconjugated polypeptide or substrate, or both.

In another embodiment, the heterologous polypeptide is purified prior to reaction with a sortase. For example, in embodiments in which one host cell is used to express the sortase, and a second host cell is employed for expression of the (secreted) heterologous polypeptide, the polypeptide can be isolated from the medium and then mixed with the first host cell expressing the sortase, along with the conjugation substrate. In such an embodiment, it can be advantageous for the heterologous polypeptide to contain an affinity tag which would facilitate its isolation. In one example, the affinity tag can be placed between the heterologous polypeptide and the sortase recognition sequence, such that upon reaction with the sortase, the affinity tag is removed in exchange for the conjugation substrate.

In some embodiments, the molecule of interest is a polypeptide and contacting the host cell with the conjugation substrate comprises adding a nucleic acid encoding the polypeptide to the culture medium such that the nucleic acid is taken up and expressed by the host cell. Methods for delivering polypeptides in the form of a nucleic acid vector encoding the polypeptide are known in the art.

Conjugated polypeptides produced by methods provided herein can be recovered from the cell culture medium using various methods known in the art. Recovering a secreted heterologous protein typically involves removal of host cells and debris from the medium, for example, by centrifugation or filtration. In cases where the protein is not secreted, protein recovery can be performed by lysing the cultured host cells, e.g., by mechanical shear, osmotic shock, or enzymatic treatment, to release the contents of the cells into the homogenate. The protein can then be separated from subcellular fragments, insoluble materials, and the like by differential centrifugation, filtration, affinity chromatography, hydrophobic interaction chromatography, ion-exchange chromatography, size exclusion chromatography, electrophoretic procedures (e.g., preparative isoelectric focusing (IEF)), ammonium sulfate precipitation, and the like. Procedures for recovering and purifying particular types of proteins are known in the art.

In an additional aspect, an isolated nucleic acid is provided herein comprising a nucleotide sequence encoding a soluble sortase operably linked to a nucleotide sequence encoding a eukaryotic signal peptide and a nucleotide sequence encoding a transmembrane domain.

In some embodiments, the isolated nucleic acid encodes a fusion protein comprising a soluble sortase operably linked to a transmembrane domain and a eukaryotic signal peptide, such that the signal peptide is capable of targeting the fusion protein for secretion by a host cell and the transmembrane domain is capable of anchoring the fusion protein in the cell membrane with the sortase exposed to the extracellular medium. The isolated nucleic acid is useful for transforming host cells such that the host cells express the soluble sortase anchored to the cell surface via the transmembrane domain.

In another aspect, an isolated nucleic acid is provided comprising a nucleotide sequence encoding a heterologous polypeptide, a nucleotide sequence encoding a sortase ligation sequence, and a nucleotide sequence encoding a eukaryotic signal peptide. In some preferred embodiments, the isolated nucleic acid encodes a fusion protein comprising a heterologous polypeptide operably linked to a sortase ligation sequence and a eukaryotic signal peptide. Where the sortase ligation sequence is a sortase recognition sequence, the sortase ligation sequence is preferably located C-terminal of the heterologous polypeptide such that cleavage and ligation of the recognition sequence by a sortase retains the heterologous polypeptide in the conjugated polypeptide.

Expression of the nucleic acid by host cells having cell surface sortase activity or host cells that are co-cultured with cells having cell surface sortase activity results in secretion of the fusion protein into the extracellular medium, where it is exposed to the cell surface sortase. Addition of a conjugation substrate comprising a molecule of interest linked to a complementary sortase ligation sequence results in ligation of the heterologous polypeptide and the molecule of interest.

In another aspect, vectors are provided comprising a nucleic acid described herein one or more additional sequences suitable for directing replication and expression of the encoded polypeptides within a host cell. Methods for isolating, replicating, and ligating DNA sequences into suitable vectors are well known in the art and are described, e.g., in Sambrook et al., Molecular Cloning: A Laboratory Manual, Cold Spring Harbor, N.Y., 1989.

In some embodiments, an isolated nucleic acid expression vector is provided herein for the expression of a fusion protein comprising an insertion site for a nucleotide sequence encoding a heterologous polypeptide operably linked to a nucleotide sequence encoding a eukaryotic signal peptide and a nucleotide sequence encoding a sortase ligation sequence, such that insertion of a nucleotide sequence into the insertion site results in an isolated nucleic acid comprising the nucleotide sequence encoding the heterologous polypeptide operably linked to both the eukaryotic signal peptide and the sortase ligation sequence. Such vectors are useful in connection with methods provided herein for conjugating a heterologous polypeptide to a molecule of interest, wherein the methods comprise inserting a nucleotide sequence encoding a heterologous polypeptide into the vector, expressing the vector in a host cell cultured in the presence of cells expressing a cell surface sortase, contacting the cultured cells expressing the cell surface sortase with a conjugation substrate, and isolating the conjugated polypeptide.

The choice of a suitable recombinant vector for use in relation to methods described herein often depends on the host cell into which the recombinant DNA is to be introduced. The vector may be an autonomously replicating vector which exists as an extra chromosomal entity and replicates independent of chromosomal replication (e.g., a plasmid), or a vector that integrates into the host cell genome and replicates together with the chromosome(s) into which it has integrated. The vector is preferably an expression vector in which coding DNA sequences, such as a DNA sequence encoding a heterologous polypeptide, are operably linked to one or more regulatory sequences designed to regulate transcription and/or translation of the DNA. The regulatory sequences are preferably derived from the same or a related species as the host cell or are otherwise designed for compatibility with the host cell. Regulatory sequences suitable for use in a variety of host cells are well known in the art and are described, e.g., herein.

Regulatory sequences useful in vectors provided herein include promoter sequences, ribosomal binding sites, transcriptional start and stop sequences, translational start and stop sequences, and enhancer or activator sequences. In some embodiments, the regulatory sequences include a promoter and transcriptional start and stop sequences.

Promoters suitable for use in mammalian host cells can include any DNA sequence capable of binding mammalian RNA polymerase and initiating downstream (3′) transcription of coding sequences of interest into mRNA. A promoter will typically have a transcription initiating region, usually located proximal to the 5′ end of the coding sequence, and a TATA box, usually located 25-30 base pairs upstream of the transcription initiation site. A promoter for use in a mammalian host cell may also contain an upstream promoter element (enhancer element), which is usually located within about 100 to 200 base pairs upstream of the TATA box and can act in either orientation.

Non-limiting examples of promoters useful in mammalian host cells include the SV40 early promoter (Subramani et al., Mol. Cell Biol. 1: 854-864 (1981)), the MT-1 (metallothionein gene) promoter (Palmiter et al., Science, 222: 809-814 (1981)), the CMV promoter (Boshart et al., Cell 41: 521-530 (1985)), the adenovirus 2 major late promoter (Kaufman and Sharp, Mol. Cell. Biol, 2: 1304-1319 (1982)), the mouse mammary tumor virus LTR promoter, and the herpes simplex virus promoter.

Examples of promoters suitable for use in yeast host cells include promoters from yeast glycolytic genes (Hitzeman et al., J. Biol. Chem. 255 (1980), 12073-12080; Alber and Kawasaki, J. Mol. Appl. Gen. 1: 419-434 (1982)) and alcohol dehydrogenase genes (Young et al., in Genetic Engineering of Microorganisms for Chemicals (Hollaender et al, eds.), Plenum Press, New York, 1982), and the TPI1 (U.S. Pat. No. 4,599,311) and ADH2-4-c (Russell et al., Nature 304: 652-654 (1983)) promoters.

Additional regulatory sequences suitable for use in mammalian host cells include a transcription termination sequence and/or a polyadenylation sequence, both of which are located 3′ to the translation stop codon. The 3′ terminus of the mature mRNA is formed by site-specific post-translational cleavage and polyadenylation. Examples of suitable transcription terminator sequences include the human growth hormone terminator (Palmiter et al., Science, 222: 809-814 (1983)), the TPI1 terminator (Alber and Kawasaki, J. Mol. Appl. Gen., 1: 419-434 (1982)) and the ADH3 terminator (McKnight et al., The EMBO J. 4, 1985, pp. 2093-2099). Examples of suitable polyadenylation sequences include the early or late polyadenylation signal from SV40 (Kaufman and Sharp, ibid.), the polyadenylation signal from the adenovirus 5 E1b region, and the human growth hormone gene terminator (DeNoto et al. Nuc. Acids Res. 9: 3719-3730 (1981)).

Vectors may also contain a set of RNA splice sites downstream from the promoter and upstream from the insertion site for the heterologous coding sequence. Preferred RNA splice sites may be obtained from adenovirus and/or immunoglobulin genes.

Expression vectors may also include a noncoding viral leader sequence, such as the adenovirus 2 tripartite leader, located between the promoter and the RNA splice sites; enhancer sequences, such as the SV40 enhancer; and a DNA sequence enabling the vector to replicate in the host cell in question, such as the SV40 origin of replication.

Expression vectors may also comprise a selectable marker, such as a gene encoding a product which complements a defect in the host cell (e.g., the gene coding for dihydrofolate reductase (DHFR) or the Schizosaccharomyces pombe TPI gene (described by P. R. Russell, Gene 40, 1985, pp. 125-130)), or a gene which confers resistance to a drug (e.g., ampicillin, kanamycin, tetracyclin, chloramphenicol, neomycin, hygromycin or methotrexate).

Integrating expression vectors also contain at least one sequence, and typically two sequences flanking the expression construct, which are homologous to a sequence of the host cell genome. The integrating vector can be directed to a specific locus in the host cell by selecting the appropriate homologous sequence for inclusion in the vector. Methods for effecting homologous recombination in mammalian host cells are described, e.g., in PCT App. Nos. US93/03868 and PCT US98/05223, each of which is incorporated herein by reference.

Selectable markers may be introduced into the cell on a separate plasmid at the same time as the sequence encoding the heterologous protein, or on the same plasmid. If on the same plasmid, the selectable marker and the gene of interest may be under the control of different promoters or the same promoter producing a dicistronic message (e.g., U.S. Pat. No. 4,713,339).

In another aspect, cells are provided comprising a nucleic acid or vector provided herein, which can be stably incorporated into the host cell genome replicating extra-chromosomally within the host cell. For example, in some embodiments, a host cell comprises an isolated nucleic acid encoding a fusion protein comprising a soluble sortase, a eukaryotic signal sequence, and a transmembrane domain, such that expression of the nucleic acid by the host cell results in secretion of the fusion protein and anchoring of the soluble sortase in the host cell membrane with the soluble sortase exposed to the extracellular medium. Such host cells are useful, e.g., in connection with an isolated nucleic acid provided herein encoding a heterologous polypeptide, a sortase ligation sequence, and a eukaryotic signal peptide, which nucleic acid can be expressed in a host cell provided herein having cell surface sortase activity such the expressed heterologous polypeptide is secreted by the host cell and the sortase cleaves and/or ligates the sortase ligation sequences of the heterologous polypeptide and a conjugation substrate to form a conjugated polypeptide.

In some embodiments, cells having cell surface-associated sortase activity are co-cultured with other host cells expressing a secreted heterologous polypeptide linked to a sortase ligation sequence. Addition of a conjugation substrate comprising a molecule of interest linked to a complementary sortase ligation sequence results in ligation of the heterologous polypeptide and the molecule of interest. In further embodiments, the heterologous polypeptide is expressed in the cells having cell surface sortase activity.

Methods of transfecting mammalian cells with recombinant DNA and expressing such DNA in the cells are described, e.g., in Kaufman and Sharp, J. Mol. Biol. 159: 601-621 (1982); Southern and Berg, J. Mol. Appl. Genet. 1: 327-341 (1982); Loyter et al., Proc. Natl. Acad. Sci. USA 79: 422-426 (1982); Wigler et al., Cell 14: 725 (1978); Corsaro and Pearson, Somatic Cell Genetics, 7: 603 (1981), Graham and van der Eb, Virology 52: 456 (1973); and Neumann et al., EMBO J. 1: 841-845 (1982). Suitable transfection methods include, but are not limited to, dextran-mediated transfection, calcium phosphate precipitation, polybrene mediated transfection, protoplast fusion, electroporation, viral infection, encapsulation of polynucleotide(s) in liposomes, and direct microinjection of the DNA into cell nuclei.

After the cells have taken up the expression vector or other recombinant DNA, they are grown in a growth medium suitable for expressing the polypeptide(s) of interest. As used herein the term “suitable growth medium” means a medium containing nutrients and other components required for the growth of host cells and the expression of polypeptides of interest. Media generally include a carbon source, a nitrogen source, essential amino acids, essential sugars, vitamins, salts, phospholipids, protein and growth factors. Drug selection is then applied to select for the growth of cells that are expressing the selectable marker in a stable fashion. For cells that have been transfected with an amplifiable selectable marker, the drug concentration may be increased to select for an increased copy number of the cloned sequences, thereby increasing expression levels.

In another aspect, compositions are provided comprising a conjugation substrate described herein. The compositions can be used to conjugate a molecule of interest associated with the conjugation substrate to a heterologous protein. Addition of a composition provided herein to cultured cells having cell surface-associated sortase activity in the presence of the heterologous polypeptide results in site-specific conjugation of the molecule of interest to the heterologous polypeptide. In some embodiments, the compositions further comprise a carrier, such as a molecule that enhances solubility, stability, and/or other characteristics of the conjugation substrate.

In another aspect, kits are provided herein for conjugating a polypeptide to a molecule of interest. In some embodiments, the kits comprise an isolated nucleic acid encoding a fusion protein comprising a soluble sortase, a eukaryotic signal sequence, and a transmembrane domain, or a vector or a cell comprising such a nucleic acid. In some embodiments, the kits further comprise an isolated nucleic acid expression vector comprising a nucleotide sequence encoding a eukaryotic signal sequence, a nucleotide sequence encoding a sortase ligation sequence, and an insertion site for inserting a nucleotide sequence encoding a heterologous polypeptide, wherein a vector comprising an inserted nucleotide sequence encodes a fusion protein comprising the heterologous polypeptide operably linked to both the sortase ligation sequence and the eukaryotic signal sequence. In further embodiments, the kits may further comprise instructions for carrying out methods provided herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the RNA effector molecules and methods featured in the invention, suitable methods and materials are described below.

All publications, patent applications, patents, and other references mentioned herein are incorporated by reference in their entirety as if each individual publication or patent or patent application was specifically and individually indicated to be incorporated by reference in its entirety. The present invention may be as defined in any one of the following numbered paragraphs:

1. An isolated nucleic acid comprising a nucleotide sequence encoding a polypeptide, the polypeptide comprising a eukaryotic signal sequence, a soluble sortase, and a transmembrane domain, wherein the signal sequence is capable of targeting the polypeptide for secretion by a eukaryotic host cell and the transmembrane domain is capable of anchoring the polypeptide in the plasma membrane of the host cell with the sortase exposed to the extracellular medium.

2. The nucleic acid of claim 1, wherein the sortase has sortase A catalytic activity.

3. The nucleic acid of any of claims 1-2 wherein the sortase is sortase A of S. aureus, or a catalytically active fragment, derivative, or variant thereof.

4. The nucleic acid of any of claims 1-3 wherein the sortase comprises residues 60-206 of sortase A of S. aureus (SEQ ID NO:2).

5. The nucleic acid of any of claims 1-4, wherein the transmembrane domain is capable of anchoring the polypeptide in the plasma membrane in a type II orientation.

6. The nucleic acid of claim 5, wherein the transmembrane domain is located N-terminal of the sortase.

7. The nucleic acid of claim 1, wherein the sortase has sortase B catalytic activity.

8. The nucleic acid of claim 1 or 7, wherein the sortase is sortase B of S. aureus, or a catalytically active fragment, derivative, or variant thereof.

9. The nucleic acid of any of claim 1 or 7-8, wherein sortase comprises residues 30-229 of sortase B of S. aureus (SEQ ID NO:4).

10. The nucleic acid of any of claim 1 or 7-9, wherein the transmembrane domain is capable of anchoring the polypeptide in the plasma membrane with a type I orientation.

11. The nucleic acid of any of claim 1 or 7-10, wherein the transmembrane domain is located C-terminal of the sortase.

12. The nucleic acid of any of claims 1-11, wherein the nucleotide sequence is operably linked to an expression control sequence.

13. The nucleic acid of any of claims 1-12, wherein the expression control sequence is a eukaryotic promoter.

14. The nucleic acid of any of claims 1-13, wherein the polypeptide further comprises an affinity tag.

15. The nucleic acid of any of claims 1-14, wherein the polypeptide further comprises a spacer peptide.

16. The nucleic acid of claim 15, wherein the spacer peptide is located between the soluble sortase and the transmembrane domain.

17. An expression vector comprising the nucleic acid of any of claims 1-16.

18. A eukaryotic cell expressing the nucleic acid of any of claims 1-17.

19. A recombinant polypeptide, comprising a eukaryotic signal sequence, a soluble sortase, and a transmembrane domain, wherein the signal sequence is capable of targeting the polypeptide for secretion by a eukaryotic host cell and the transmembrane domain is capable of anchoring the polypeptide in the plasma membrane of the host cell with the sortase exposed to the extracellular medium.

20. The recombinant polypeptide of claim 19, further comprising an affinity tag.

21. A recombinant polypeptide, comprising a eukaryotic signal sequence, a heterologous polypeptide, and a sortase ligation sequence, wherein the signal sequence is capable of targeting the polypeptide for secretion by a eukaryotic host cell.

22. The recombinant polypeptide of claim 21, further comprising an affinity tag.

23. The recombinant polypeptide of any of claims 21-22, wherein the sortase ligation sequence comprises a sortase recognition sequence.

24. The recombinant polypeptide of claim 23, wherein the sortase ligation sequence is located C-terminal of the heterologous polypeptide.

25. The recombinant polypeptide of claim 23, wherein the sortase recognition sequence is a sortase A recognition sequence having the consensus sequence X₁PX₂X₃G (SEQ ID NO:5), wherein X₁ is Leu, Ile, Val or Met, P is Pro, X₂ is any amino acid, X₃ is Ser, Thr or Ala, and G is Gly.

26. The recombinant polypeptide of claim 25, wherein X₂ is Asp, Glu, Ala, Gln, Lys or Met.

27. The recombinant polypeptide of claim 25, wherein sortase A recognition sequence has the consensus sequence LPXTG (SEQ ID NO:6), wherein L is Leu, P is Pro, X is any amino acid, T is Thr, and G is Gly.

28. The recombinant polypeptide of claim 23, wherein the sortase recognition sequence is a sortase B recognition sequence having the consensus sequence NPX₁TX₂ (SEQ ID NO:7), wherein N is Asn, P is Pro, X₁ is Gln or Lys, T is Thr, and X₂ is Asp or Gly.

29. The recombinant polypeptide of claim 28, wherein the sortase B recognition sequence is NPQTN (SEQ ID NO:8).

30. The recombinant polypeptide of claim 21, wherein the sortase ligation sequence comprises a polyglycine sequence.

31. The recombinant polypeptide of claim 30, wherein the polyglycine sequence comprises 1, 2, 3, 4 or 5 glycine residues.

32. The recombinant polypeptide of claim 30, wherein the sortase ligation sequence is located N-terminal of the heterologous polypeptide.

33. The recombinant polypeptide of claim 32, wherein the sortase ligation sequence is located C-terminal of the signal sequence.

34. An expression vector comprising a nucleotide sequence encoding a fusion protein, the fusion protein comprising a heterologous polypeptide, a eukaryotic signal sequence capable of targeting the fusion protein for secretion by a eukaryotic host cell, and a sortase ligation sequence.

35. The expression vector of claim 34, wherein the sortase ligation sequence comprises a sortase recognition sequence.

36. The expression vector of claim 35, wherein the sortase ligation sequence is located C-terminal of the heterologous polypeptide.

37. The expression vector of claim 35, wherein the sortase recognition sequence is a sortase A recognition sequence having the consensus sequence X₁PX₂X₃G (SEQ ID NO:5), wherein X₁ is Leu, Ile, Val or Met, P is Pro, X₂ is any amino acid, X₃ is Ser, Thr or Ala, and G is Gly.

38. The expression vector of claim 37, wherein X₂ is Asp, Glu, Ala, Gln, Lys or Met.

39. The expression vector of claim 37, wherein sortase A recognition sequence has the consensus sequence LPXTG (SEQ ID NO:6), wherein L is Leu, P is Pro, X is any amino acid, T is Thr, and G is Gly.

40. The expression vector of claim 35, wherein the sortase recognition sequence is a sortase B recognition sequence having the consensus sequence NPX₁TX₂ (SEQ ID NO:7), wherein N is Asn, P is Pro, X₁ is Gln or Lys, T is Thr, and X₂ is Asp or Gly.

41. The expression vector of claim 40, wherein the sortase B recognition sequence is NPQTN (SEQ ID NO:8).

42. The expression vector of claim 34, wherein the sortase ligation sequence comprises a polyglycine sequence.

43. The expression vector of claim 42, wherein the polyglycine sequence comprises 1, 2, 3, 4 or 5 glycine residues.

44. The expression vector of claim 42, wherein the sortase ligation sequence is located N-terminal of the heterologous polypeptide. 45. A method for producing a conjugated polypeptide comprising:

a) expressing a first nucleotide sequence encoding a first fusion protein in a cultured host cell, the first fusion protein comprising a first eukaryotic signal sequence, a transmembrane domain and a soluble sortase, wherein the first signal sequence targets the first fusion protein for secretion by the host cell and the transmembrane domain anchors the first fusion protein in the plasma membrane of the cell with the sortase exposed to the extracellular medium;

b) expressing a second nucleotide sequence encoding a second fusion protein in a cultured host cell, the second fusion protein comprising a second eukaryotic signal sequence, a heterologous polypeptide, and a first sortase ligation sequence, wherein the second signal sequence targets the second fusion protein for secretion by the host cell;

c) ontacting the cell with a conjugation substrate comprising a second sortase ligation sequence and a molecule of interest, wherein one of the first or second sortase ligation sequences comprises a sortase recognition sequence and the other of the first or second sortase ligation sequences comprises a polyglycine sequence;

d) maintaining the cell under conditions which allow the sortase to cleave the sortase recognition sequence and ligate the cleaved sortase recognition sequence to the polyglycine sequence to form a conjugated polypeptide; and

e) isolating the conjugated polypeptide.

46. A method of claim 45, wherein the first or second sortase ligation sequence comprises a sortase A recognition sequence having the consensus sequence X₁PX₂X₃G, wherein the second signal sequence targets the second fusion protein for secretion by the host cell;

contacting the cell with a conjugation substrate comprising a second sortase ligation sequence and a molecule of interest, wherein one of the first or second sortase ligation sequences comprises a sortase recognition sequence and the other of the first or second sortase ligation sequences comprises a polyglycine sequence;

maintaining the cell under conditions which allow the sortase to cleave the sortase recognition sequence and ligate the cleaved sortase recognition sequence to the polyglycine sequence to form a conjugated polypeptide; and

isolating the conjugated polypeptide.

47. The method of claim 46, wherein the ligation of the cleaved sortase recognition sequence includes formation of an amide bond between a C-terminal carboxyl group of the cleaved sortase recognition sequence and an N-terminal amino group of the polyglycine sequence.

48. The method of claim 46, wherein the first sortase ligation sequence comprises the sortase recognition sequence and the second sortase ligation sequence comprises the polyglycine sequence.

49. The method of claim 48, wherein the sortase recognition sequence is located C-terminal of the heterologous polypeptide.

50. The method of claim 48, wherein the second fusion protein further comprises an affinity tag located C-terminal of the sortase ligation sequence, the affinity tag being cleaved from the conjugated polypeptide.

51. The method of claim 48, wherein the second fusion protein further comprises an affinity tag located N-terminal of the sortase ligation sequence, the affinity tag being retained in the conjugated polypeptide.

52. The method of claim 48, wherein the conjugation substrate further comprises an affinity tag located C-terminal of the second sortase ligation sequence, the affinity tag being retained in the conjugated polypeptide.

53. The method of claim 46, wherein the first sortase ligation sequence comprises the polyglycine sequence and the second sortase ligation sequence comprises the sortase recognition sequence.

54. The method of claim 53, wherein the second eukaryotic signal sequence is at the N-terminus of the second fusion protein and the polyglycine sequence is located C-terminal of the affinity tag.

55. The method of claim 54, wherein the second eukaryotic signal sequence is capable of being cleaved by a host cell enzyme, wherein the polyglycine sequence is located at the N-terminus of the second fusion protein upon cleavage of the second eukaryotic signal sequence.

56. The method of claim 53, wherein the sortase recognition sequence is located C-terminal of the molecule of interest.

57. The method of claim 53, wherein the conjugation substrate further comprises an affinity tag located N-terminal of the second sortase ligation sequence, the affinity tag being retained in the conjugated polypeptide.

58. The method of claim 53, wherein the conjugation substrate further comprises an affinity tag located C-terminal of the second sortase ligation sequence, the affinity tag being cleaved from the conjugated polypeptide.

59. The method of claim 53, wherein the second fusion protein further comprises an affinity tag located C-terminal of the first sortase ligation sequence, the affinity tag being retained in the conjugated polypeptide.

60. The method of claim 46, wherein the sortase recognition sequence is a sortase A recognition sequence having the consensus sequence X₁PX₂X₃G (SEQ ID NO:5), wherein X₁ is Leu, Ile, Val or Met, P is Pro, X₂ is any amino acid, X₃ is Ser, Thr or Ala, and G is Gly.

61. The method of claim 60, wherein X₂ is Asp, Glu, Ala, Gln, Lys or Met.

62. The method of claim 60, wherein sortase A recognition sequence has the consensus sequence LPXTG (SEQ ID NO:6), wherein L is Leu, P is Pro, X is any amino acid, T is Thr, and G is Gly.

63. The method of claim 46, wherein the sortase recognition sequence is a sortase B recognition sequence having the consensus sequence NPX₁TX₂ (SEQ ID NO:7), wherein N is Asn, P is Pro, X₁ is Gln or Lys, T is Thr, and X₂ is Asp or Gly.

64. The method of claim 63, wherein the sortase B recognition sequence is NPQTN (SEQ ID NO:8).

65. The method of claim 46, wherein the polyglycine sequence comprises 1, 2, 3, 4 or 5 glycine residues.

66. The method of claim 46, wherein the first fusion protein further comprises a spacer peptide.

67. The method of claim 66, wherein the spacer peptide is located between the sortase and the transmembrane domain.

68. The method of claim 46, wherein the second fusion protein further comprises a spacer peptide.

69. The method of claim 68, wherein the spacer peptide is located between the heterologous polypeptide and the first sortase ligation sequence.

70. The method of claim 46, wherein the first and/or second eukaryotic signal sequences are capable of being cleaved by a host cell enzyme.

71. The method of claim 46, wherein the conjugation substrate is of the formula S-L-R, where S is the second sortase ligation sequence, L is an optional linker and R is the molecule of interest.

72. The method of claim 71, wherein R or L comprises a water-soluble, non-peptidic polymer with an average molecular weight of about 200 to about 100,000 Daltons.

73. The method of claim 72, wherein the polymer is a poly(ethylene glycol) (PEG) or a methoxypoly(ethylene glycol) (mPEG).

74. The method of claim 71, wherein R is selected from the group consisting of: silane, fluorescein, rhodamine, FITC and biotin.

75. The method of claim 71, wherein L is a hydrolytically stable linker.

76. The method of claim 71, wherein L comprises at least 3 contiguous saturated carbon atoms.

77. A composition comprising a conjugation substrate of the formula S-L-R, where S is the second sortase ligation sequence, L is an optional linker and R is the molecule of interest.

78. The composition of claim 77, wherein R is a water-soluble, non-peptidic polymer with an average molecular weight of about 200 to about 100,000 Daltons.

79. The composition of claim 78, wherein the polymer is a poly(ethylene glycol) (PEG) or a methoxypoly(ethylene glycol) (mPEG).

80. The composition of claim 77, wherein L is a hydrolytically stable linker.

81. The composition of claim 77, wherein L comprises at least 3 contiguous saturated carbon atoms.

82. The composition of claim 77, wherein the sortase ligation sequence comprises a sortase A recognition sequence having the consensus sequence X₁PX₂X₃G (SEQ ID NO:5), wherein X₁ is Leu, Ile, Val or Met, P is Pro, X₂ is any amino acid, X₃ is Ser, Thr or Ala, and G is Gly.

83. The composition of claim 82, wherein X₂ is Asp, Glu, Ala, Gln, Lys or Met.

84. The composition of claim 82, wherein the sortase ligation sequence comprises a sortase A recognition sequence having the consensus sequence LPXTG (SEQ ID NO:6), wherein L is Leu, P is Pro, X is any amino acid, T is Thr, and G is Gly.

85. The composition of claim 77, wherein the sortase ligation sequence comprises a sortase B recognition sequence having the consensus sequence NPX₁TX₂ (SEQ ID NO:7), wherein N is Asn, P is Pro, X₁ is Gln or Lys, T is Thr, and X₂ is Asp or Gly.

86. The composition of claim 85, wherein the sortase B recognition sequence is NPQTN (SEQ ID NO:8).

87. The composition of claim 77, wherein the sortase ligation sequence comprises a polyglycine sequence.

88. The composition of claim 87, wherein the polyglycine sequence comprises 1, 2, 3, 4 or 5 glycine residues.

The materials, methods, and examples are illustrative only and not intended to be limiting. Many modifications and variations of this invention can be made without departing from its spirit and scope, as will be apparent to those skilled in the art. The specific embodiments described herein are offered by way of example only, and the invention is to be limited only in terms of the appended claims, along with the full scope of equivalents to which such claims are entitled.

### EXAMPLES

### Example 1

### Assay for Measuring Rate of Sortase-Mediated Cleavage and Ligation.

To assay sortase peptide-peptide ligation activity, a soluble sortase (10 μM SPA in buffer containing 50 mM Tri-HCl, pH 7.5, 150 mM NaCl, 5 mM CaCl₂, and 2 mM BME) is incubated with a fluorescent peptide substrate [acetyl-RE(Edans)LPKTGK(Dabcyl)R (SEQ ID NO:9)] comprising a sortase consensus recognition sequence conjugated to a fluorophore that allows the rate of substrate cleavage to be measured as a fluorescence increase at an emission wavelength of 460 nm and an excitation wavelength of 360 nm on a fluorometer (Applied Biosystems CYTOFLUOR Series 4000). The sortase and the fluorescent peptide substrate are incubated with a series of peptides comprising a polyglycine sequence (GₙRRNRRTSKLMR (SEQ ID NO:10), where n is 1, 2, 3 or 5). Product formation is monitored by a C-18 reverse phase HPLC over the course of 28 hrs, using a gradient of 0.5% to 38% CH₃CN in 0.1% trifluoroacetic acid in 40 minutes at a flow rate of 1 ml/min. Elution of peptides is monitored at 214 nm and fractions are collected for mass analysis on a MALDI-TOF mass spectrometer.

To assay sortase protein-peptide ligation activity, a protein substrate (GFP-LPXTG-6His (SEQ ID NO:11) or GST-LPXTG-6His (SEQ ID NO:12)) comprising a sortase recognition sequence conjugated to a reporter protein is incubated at concentrations ranging 10 μM to 35 μM with a soluble sortase (10 μM SPA in buffer containing 50 mM Tri-HCl, pH 7.5, 150 mM NaCl, 5 mM CaCl₂, and 2 mM BME) and a series of peptides comprising an N-terminal polyglycine sequence (GₙRRNRRTSKLMR (SEQ ID NO:10), where n is 1, 2, 3 or 5) added in 5 to 10-fold excess. The reactions are incubated at 37° C. for 24 to 48 hours, and terminated by passing the reaction mixtures through a 0.5 ml Ni-NTA column equilibrated with 50 mM Tris-HCl pH 7.5 and 150 mM NaCl. The protein ligation product is collected in the column flow through, which is further purified on a 10DG desalting column to remove the unligated peptide.

### Example 2

### Hydrolysis of LPXTG-Motif Containing Proteins In Vitro

To determine the hydrolysis efficiency of a sortase on proteins, the sortase is incubated with two different LPXTG containing substrates (GST-LPXTG-6His (SEQ ID NO:12) and GFP-LPXTG-6His (SEQ ID NO:11)) and the cleavage products are analyzed by SDS/PAGE and MALDI-TOF mass spectroscopy.

### Example 3

### Ligation with LPXTG-Containing Peptides and Proteins In Vitro

In addition to hydrolysis, sortase catalyzed transpeptidation is effected in vitro in the presence of a tripeptide (Gly)₃. The native conjugation partner for LPXTG-containing protein in vivo is a pentaglycine cross bridge on cell walls. The formation of the ligation product RE (Edans) LPKTGₙRRNRRTSKLMLR (n=1, 2, 3, or 5) (SEQ ID NO:13) by RP-HPLC and mass spectrometry analyses is determined

The sortase-mediated ligation method is also applied to protein-peptide conjugation. Protein GFP-LPXTG-6His (SEQ ID NO:11) and a ten-fold excess of the peptide GGGGGRRNRRTSKLMLR (SEQ ID NO:14) are mixed and incubated in the presence of different amount of sortase. Product formation is monitored by SDS/PAGE and MALDI-TOF mass spectrometry.

### Example 4

### Conjugation of NH₂—CH₂— Containing Compounds to LPXTG Substrates

Sortase activity is tested further with non-peptidyl substrates. Since an N-terminal glycine rather than amino acids with a branched alpha-carbon facilitates nucleophilic attack, it is possible that sortase might accommodate a substrate with a NH₂—CH₂-group. A protein substrate (GFP-LPXTG-6His (SEQ ID NO:11)) is incubated with sortase in the presence of 5 mM glycine, 5 mM spermine (Sigma), 0.5 mM 3.4 kDa poly (ethylene glycol)-ω-amino-α-carboxyl (NH₂-PEG-COOH) (Shearwater), or 0.5 mM peptide-1 (GₙRRNRRTSKLMLR (SEQ ID NO:10), where n=1, 3, or 5) in ligation buffer. After 20 hours at 37° C., the ligation reactions are analyzed on a NOVEX 4-12% Bis-Tris gel with MES running buffer. The molecular weights of the ligation products are also determined by MALDI-TOF mass spectroscopy and the ligation efficiencies are compared.

### Example 5

### Utilization of a Sortase Variant in Protein Ligation Processes

Nucleic acids encoding sortase B are prepared and isolated according to processes described in Mazmanian et al., Proc. Natl. Acad. Sci. USA 99: 2293-2298 (2002) and U.S. Pat. No. 7,101,692 and references cited therein, all of which are herein incorporated by reference. Sortase B is utilized in the processes described in Examples 2-4, with target proteins and peptides having a NPX₁TX₂ recognition sequence, where X₁ is glutamine or lysine; X₂ is asparagine or glycine; N is asparagine; P is proline and T is threonine (SEQ ID NO:7).

### Example 6

### PEGylation of β-Interferon

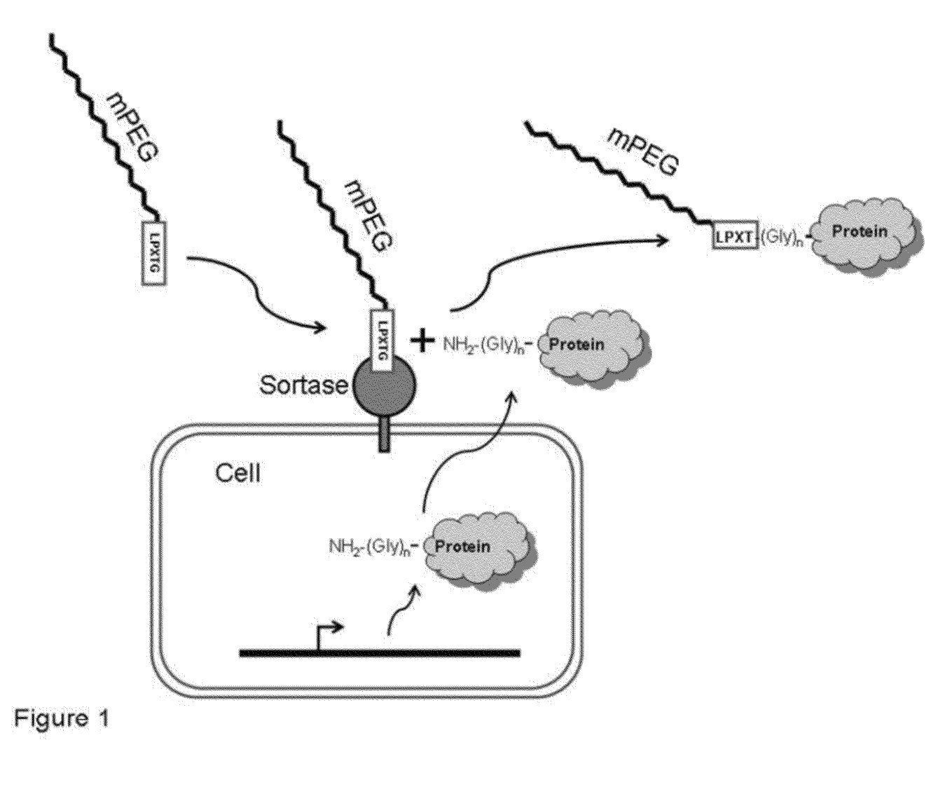

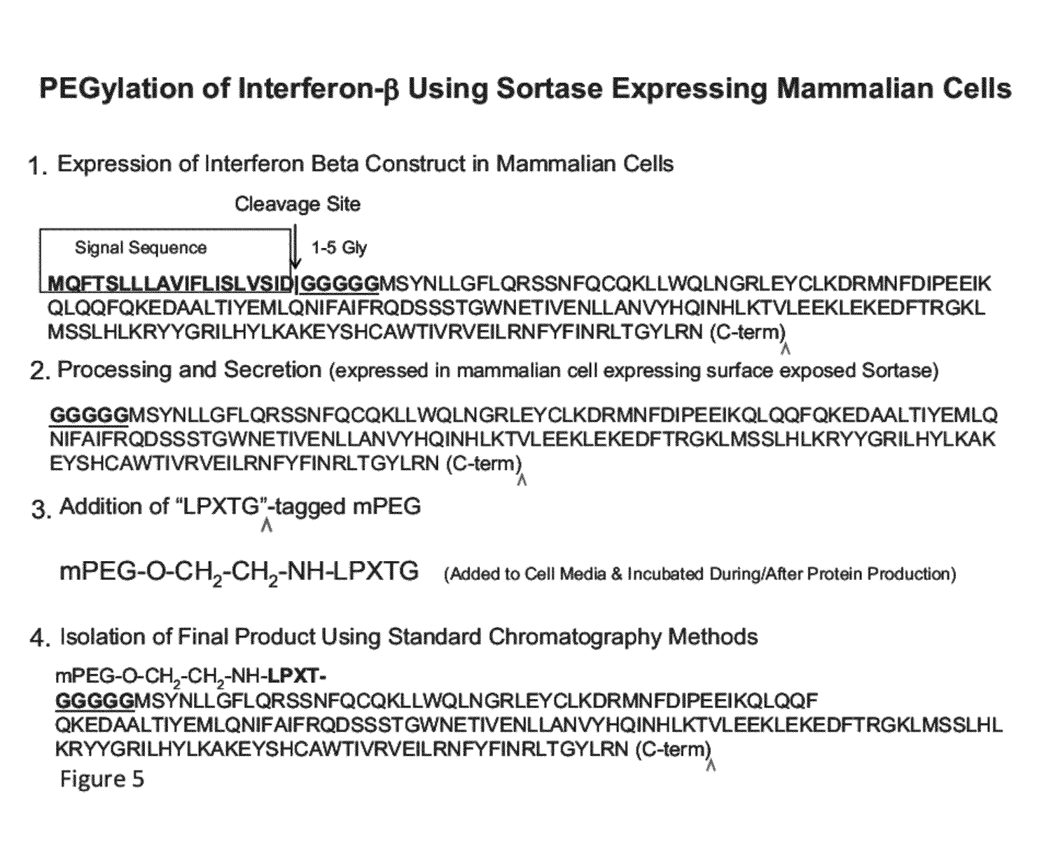

Mammalian cells (FIG. 1) are transformed with an expression vector encoding a β-interferon fusion protein (NH₂-(Gly)ₙ-Protein in FIG. 1). The fusion protein (SEQ ID NO:15; FIG. 5, construct 1) comprises β-interferon (SEQ ID NO:16) linked at the N-terminus to a polyglycine sequence (SEQ ID NO:17) and a signal peptide (SEQ ID NO:18). The mammalian cells are also transformed with an expression vector encoding a second fusion protein comprising a sortase having sortase A catalytic activity, a signal peptide, and a transmembrane domain. Upon expression of the second fusion protein, the signal peptide and the transmembrane domain target the second fusion protein for secretion by the mammalian cells and retention in the plasma membrane, such that the cells express a surface-associated sortase with the sortase catalytic domain exposed to the extracellular medium (FIG. 1). Alternatively, the mammalian cells expressing the β-interferon fusion protein can be cultured in the presence of a separate population of mammalian cells expressing the sortase fusion protein.

The transformed mammalian cells are cultured in a bioreactor under conditions suitable for expression of the fusion proteins. A conjugation substrate (mPEG-LPXTG in FIG. 1) is added to the culture medium near the end of the log phase growth cycle (e.g., around day 6-8). The conjugation substrate (SEQ ID NO:19; FIG. 5, construct 3) comprises a sortase A recognition sequence (LPXTG, where L is Leu, P is Pro, X is any amino acid, T is Thr, and G is Gly (SEQ ID NO:6)) linked to a molecule of interest comprising a 5K to 40K single chain or branched chain mPEG polymer.

The cells are incubated with the conjugation substrate at 37° C. for 10-14 days in the bioreactor; during this time, the sortase fusion protein is expressed and translocated to the cell surface such that the sortase is associated with the cell surface and the sortase catalytic domain is exposed to the extracellular medium. The β-interferon fusion protein (SEQ ID NO:15; FIG. 5, construct 1) is also expressed, the signal peptide is removed and the truncated polypeptide with an N-terminal polyglycine sequence (SEQ ID NO:20; FIG. 5, construct 2) is secreted. Incubation of the surface-associated sortase in the presence of the sortase substrates (i.e., the secreted β-interferon and the conjugation substrate) in the extracellular medium results in sortase-catalyzed cleavage of the conjugation substrate within the sortase recognition sequence and sortase-catalyzed ligation of the cleaved conjugation substrate to the N-terminal glycine of the β-interferon fusion protein, resulting in the formation of a mPEG-β-interferon conjugate (SEQ ID NO:21; FIG. 5, construct 4). The PEGylated β-interferon is then isolated using standard chromatography methods.

### Additional Specification Sequences:

Nucleotide Sequence for Sortase A (Sa-SrtA) Stapylococcus aureus (SEQ ID NO:1)

| LOCUS | AF162687 1256 bp DNA linear BCT 11-AUG-1999 |
|---|---|
| DEFINITION | Staphylococcus aureus sortase (srtA) gene, complete cds. |
| ACCESSION | AF162687 |
| VERSION | AF162687.1 GI: 5726435 |
| KEYWORDS | . |
| SOURCE | Staphylococcus aureus |
| ORGANISM | Staphylococcus aureus Bacteria; Firmicutes; |
| | Bacillales; Staphylococcus. |
| REFERENCE | 1 (bases 1 to 1256) |
| AUTHORS | Mazmanian,S. K., Liu, G., Ton-That, H. and Schneewind, O. |
| TITLE | Staphylococcus aureus sortase, an enzyme that anchors |
| | surface proteins to the cell wall |
| JOURNAL | Science 285 (5428), 760-763 (1999) |
| PUBMED | 10427003 |
| REFERENCE | 2 (bases 1 to 1256) |
| AUTHORS | Mazmanian, S. K., Liu, G., Ton-That, H. and Schneewind, O. |
| TITLE | Direct Submission |
| JOURNAL | Submitted (24-JUN-1999) Microbiology and Immunolgy, UCLA, |
| | 10833 Le Conte Avenue, Los Angeles, CA 90095, USA |
| FEATURES | Location/Qualifiers |
| source | 1 . . . 1256 |
| | /organism = “Staphylococcus aureus” |
| | /mol_type = “genomic DNA” |
| | /strain = “8325-4” |
| | /db_xref = “taxon: 1280” |
| gene | 483 . . . 1103 |
| | /gene = “srtA” |
| CDS | 483 . . . 1103 |
| | /gene = “srtA” |
| | /note = “transpeptidase” |
| | /codon_start = 1 |
| | /transl_table = 11 |
| | /product = “sortase” |
| | /protein_id = “AAD48437.1” |
| | /db_xref = “GI: 5726436” |
| ORIGIN | |
| (SEQ ID NO: 1) | |
| 1 | tagcaatacc ttttcctcta gctgaagcat cgacataaat agaatgttcg attgtatata |
| 61 | ggtatgctgg ccaaggtcta aatgaaccga acgtcgcaaa ccctaagaca cttccatttt |
| 121 | cctcaaatac aaagataggc tcatgcttac gttgtttcgt ttcaaaccat gcgacacgtt |
| 181 | cgtctatggt ttgtggttca taagtataaa cagctgtagt attgataatg gcatcattgt |
| 241 | atatcgctaa tatagcgttt aaatcctctt ttttagcgta tctaatcata tcaattcccc |
| 301 | cttagtaatt attaaaagcg tttcgttatt tgaatgcaaa tatgtgtaat gaaatctaac |
| 361 | gtaaaagtat acatgtaaat tttatagtat aaaatgaatt gctatgagtc attttgaaat |
| 421 | taatggtata ctatatgaaa tgttaacagg cattgtgaaa tgtataaaag gagccttaac |
| 481 | gtatgaaaaa atggacaaat cgattaatga caatcgctgg tgtggtactt atcctagtgg |
| 541 | cagcatattt gtttgctaaa ccacatatcg ataattatct tcacgataaa gataaagatg |
| 601 | aaaagattga acaatatgat aaaaatgtaa aagaacaggc gagtaaagat aaaaagcagc |
| 661 | aagctaaacc tcaaattccg aaagataaat cgaaagtggc aggctatatt gaaattccag |
| 721 | atgctgatat taaagaacca gtatatccag gaccagcaac acctgaacaa ttaaatagag |
| 781 | gtgtaagctt tgcagaagaa aatgaatcac tagatgatca aaatatttca attgcaggac |
| 841 | acactttcat tgaccgtccg aactatcaat ttacaaatct taaagcagcc aaaaaaggta |
| 901 | gtatggtgta ctttaaagtt ggtaatgaaa cacgtaagta taaaatgaca agtataagag |
| 961 | atgttaagcc tacagatgta ggagttctag atgaacaaaa aggtaaagat aaacaattaa |
| 1021 | cattaattac ttgtgatgat tacaatgaaa agacaggcgt ttgggaaaaa cgtaaaatct |
| 1081 | ttgtagctac agaagtcaaa taatctatta cgctaatgga tgaatatatt gagtggaaaa |
| 1141 | cagtcttgat tgcgagactg ttttttgttt ggtatgaggt agcaatgacg acgtgtcatt |
| 1201 | ggtggagatt gtaaaaatac ataataaaaa gaagcggcaa tgtataccgc tccttt |

Protein Sequence for Sortase A (Sa-SrtA) Stapylococcus aureus (SEQ ID NO:2)

| LOCUS | AF162687_1 206 aa linear BCT 11-AUG-1999 |
|---|---|
| DEFINITION | sortase [Staphylococcus aureus]. |
| ACCESSION | AAD48437 |
| VERSION | AAD48437.1 GI: 5726436 |
| DBSOURCE | accession AF162687.1 |
| KEYWORDS | . |
| SOURCE | Staphylococcus aureus |
| ORGANISM | Staphylococcus aureus Bacteria; |
| | Firmicutes; Bacillales; Staphylococcus. |
| REFERENCE | 1 (residues 1 to 206) |
| AUTHORS | Mazmanian, S. K., Liu, G., Ton-That, H. and Schneewind, O. |
| TITLE | Staphylococcus aureus sortase, an enzyme that anchors |
| | surface proteins to the cell wall |
| JOURNAL | Science 285 (5428), 760-763 (1999) |
| PUBMED | 10427003 |
| REFERENCE | 2 (residues 1 to 206) |
| AUTHORS | Mazmanian, S. K., Liu, G., Ton-That, H. and Schneewind, O. |
| TITLE | Direct Submission |
| JOURNAL | Submitted (24-JUN-1999) Microbiology and Immunolgy, UCLA, |
| | 10833 Le Conte Avenue, Los Angeles, CA 90095, USA |
| COMMENT | Method: conceptual translation. |
| FEATURES | Location/Qualifiers |
| source | 1 . . . 206 |
| | /organism = “Staphylococcus aureus” |
| | /strain = “8325-4” |
| | /db_xref = “taxon: 1280” |
| Protein | 1 . . . 206 |
| | /product = “sortase” |
| | /name = “transpeptidase” |
| Region | 71 . . . 203 |
| | /region_name = “Sortase” |
| | /note = “Sortases are cysteine transpeptidases, |
| | found in gram-positive bacteria, that anchor surface |
| | proteins to peptidoglycans of the bacterial cell wall |
| | envelope. They do so by catalyzing a transpeptidation |
| | reaction in which the surface protein substrate |
| | is . . .; cd00004” |
| | /db_xref = “CDD: 99708” |
| Site | order(93, 105, 116, 118, 120, 183 . . . 184, 194, 197) |
| | /site type = “active” |
| | /db_xref = “CDD: 99708” |
| Site | order(120, 184, 197) |
| | /site_type = “other” |
| | /note = “catalytic site” |
| | /db_xref = “CDD: 99708” |
| CDS | 1 . . . 206 |
| | /gene = “srtA” |
| | /coded_by = “AF162687.1: 483 . . . 1103” |
| | /transl_table = 11 |
| ORIGIN | |
| (SEQ ID NO: 2) | |
| 1 | mkkwtnrlmt iagvvlilva aylfakphid nylhdkdkde kieqydknvk eqaskdkkqq |
| 61 | akpqipkdks kvagyieipd adikepvypg patpeqlnrg vsfaeenesl ddqnisiagh |
| 121 | tfidrpnyqf tnlkaakkgs mvyfkvgnet rkykmtsird vkptdvgvld eqkgkdkqlt |
| 181 | litcddynek tgvwekrkif vatevk |

Nucleotide Sequence for Sortase B (Sa-SrtB) Stapylococcus aureus (SEQ ID NO:3)

| LOCUS | BA000033 735 bp DNA linear BCT 21-DEC-2007 |
|---|---|
| DEFINITION | Staphylococcus aureus subsp. aureus MW2 DNA, |
| | complete genome. |
| ACCESSION | BA000033 REGION: 1113163 . . . 1113897 |
| VERSION | BA000033.2 GI: 47118312 |
| DBLINK | Project: 306 |
| KEYWORDS | . |
| SOURCE | Staphylococcus aureus subsp. aureus MW2 |
| ORGANISM | Staphylococcus aureus subsp. aureus MW2 |
| | Bacteria; Firmicutes; Bacillales; Staphylococcus. |
| REFERENCE | 1 |
| AUTHORS | Baba, T., Takeuchi, F., Kuroda, M., Yuzawa, H., |
| | Aoki, K., Oguchi, A., Nagai, Y., Iwama, N., |
| | Asano, K., Naimi, T., Kuroda, H., Cui, L., |
| | Yamamoto, K. and Hiramatsu, K. |
| TITLE | Genome and virulence determinants of high virulence |
| | community-acquired MRSA |
| JOURNAL | Lancet 359 (9320), 1819-1827 (2002) |
| PUBMED | 1204478 |
| REFERENCE | 2 (bases 1 to 735) |
| AUTHORS | Aoki, K., Oguchi, A., Nagai, Y., Asano, K., |
| | Iwama, N., Baba, T., Kuroda, M., Hiramatsu, K. |
| | and Kikuchi, H. |
| TITLE | Direct Submission |
| JOURNAL | Submitted (06-MAR-2002) Contact: Director-General, |
| | Biotechnology Center National Institute of |
| | Technology and Evaluation, Biotechnology |
| | Center; 2Chome 49-10 Nishihara, Shibuya-ku, |
| | Tokyo 151-0066, Japan URL :http://www.bio.nite.go.jp/ |
| COMMENT | On or before Nov. 5, 2004 this sequence version replaced |
| | gi: 21203164, gi: 21203407, gi: 21203693, gi: 21203989, |
| | gi: 21204263, gi: 21204509, gi: 21204850, gi: 21205117, |
| | gi: 21205425, gi: 21205708. |
| FEATURES | Location/Qualifiers |
| source | 1 . . . 735 |
| | /organism = “Staphylococcus aureus subsp. aureus |
| | MW2” |
| | /mol_type = “genomic DNA” |
| | /strain = “MW2” |
| | /sub_species = “aureus” |
| | /db_xref = “taxon: 196620” |
| gene | 1 . . . 735 |
| | /gene = “srtB” |
| CDS | 1 . . . 735 |
| | /gene = “srtB” |
| | /note = “ORFID: MW1017” |
| | /codon_start = 1 |
| | /transl_table = 11 |
| | /product = “NPQTN specific sortase B” |
| | /protein_id = “BAB94882.1” |
| | /db_xref = “GI: 21204184” |
| ORIGIN | |
| (SEQ ID NO: 3) | |
| 1 | atgagaatga agcgattttt aactattgta caaattttat tggttgtaat tattatcatt |
| 61 | tttggttaca aaattgttca aacatatatt gaagacaagc aagaacgcgc aaattatgag |
| 121 | aaattacaac aaaaatttca aatgctgatg agcaaacatc aagcacatgt gagaccacaa |
| 181 | tttgaatcac ttgaaaaaat aaataaagac attgttggat ggataaaatt atcaggaaca |
| 241 | tcattaaatt atccagtact acaaggtaag acaaatcacg attatttaaa tttagatttt |
| 301 | gagcgagaac atcgacgtaa aggtagtatt tttatggatt ttagaaatga attgaagaat |
| 361 | ttaaatcata atactatttt atacgggcac catgtcggtg ataatacgat gtttgatgtg |
| 421 | ttagaagatt atttaaagca atcgttttat gaaaaacaca agataattga atttgacaat |
| 481 | aaatatggta aatatcaatt gcaagtattt agtgcatata aaactactac taaagataat |
| 541 | tacatacgta cagattttga aaatgatcaa gattatcaac aatttttaga tgaaacaaaa |
| 601 | cgtaaatctg taattaattc agatgttaat gtaacggtaa aagatagaat aatgacttta |
| 661 | tcaacgtgcg aagatgcata tagtgaaaca acgaaaagaa ttgttgttgt cgcaaaaata |
| 721 | attaaggtaa gttaa |

Protein Sortase B (Sa-SrtB) Stapylococcus aureus (SEQ ID NO:4)

| LOCUS | NP_645834 244 aa linear BCT 31-MAR-2010 |
|---|---|
| DEFINITION | NPQTN specificsortase B [Staphylococcus aureus subsp. |
| | aureus MW2]. |
| ACCESSION | NP_645834 |
| VERSION | NP_645834.1 GI: 21282746 |
| DBLINK | Project: 57903 |
| DBSOURCE | REFSEQ: accession NC_003923.1 |
| KEYWORDS | . |
| SOURCE | Staphylococcus aureus subsp. aureus MW2 |
| ORGANISM | Staphylococcus aureus subsp. aureus MW2 |
| | Bacteria; Firmicutes; Bacillales; Staphylococcus. |
| REFERENCE | 1 |
| AUTHORS | Baba, T., Takeuchi, F., Kuroda, M., Yuzawa, H., |
| | Aoki, K., Oguchi, A., Nagai, Y., Iwama, N., |
| | Asano, K., Naimi, T., Kuroda, H., Cui, L., |
| | Yamamoto, K. and Hiramatsu, K. |
| TITLE | Genome and virulence determinants of high virulence |
| | community-acquired MRSA |
| JOURNAL | Lancet 359 (9320), 1819-1827 (2002) |
| PUBMED | 1204478 |
| REFERENCE | 2 (residues 1 to 244) |
| CONSRTM | NCBI Genome Project |
| TITLE | Direct Submission |
| JOURNAL | Submitted (31-MAY-2002) National Center for Biotechnology |
| | Information, NIH, Bethesda, MD 20894, USA |
| REFERENCE | 3 (residues 1 to 244) |
| AUTHORS | Aoki, K., Oguchi, A., Nagai,Y., Asano, K., Iwama, N., |
| | Baba, T., Kuroda, M., Hiramatsu, K. and Kikuchi, H. |
| TITLE | Direct Submission |
| JOURNAL | Submitted (06-MAR-2002) Biotechnology Center, National |
| | Institute of Technology and Evaluation, 2Chome 49-10 |
| | Nishihara, Shibuya-ku, Tokyo 151-0066, Japan |
| COMMENT | PROVISIONAL REFSEQ: This record has not yet been subject |
| | to final NCBI review. The reference sequence was derived |
| | from RAB94882. |
| Method: | conceptual translation. |
| FEATURES | Location/Qualifiers |
| source | 1 . . . 244 |
| | /organism = “Staphylococcus aureus subsp. aureus |
| | MW2” |
| | /strain = “MW2” |
| | /db_xref = “taxon: 196620” |
| Protein | 1 . . . 244 |
| | /product = “NPQTN specific sortase B” |
| | /calculated_mol_wt = 28974 |
| Region | 32 . . . 229 |
| | /region_name = “Sortase_B_2” |
| | /note = “Sortase B (SrtB) or subfamily-2 sortases |
| | are membrane cysteine transpeptidases found in gram- |
| | positive bacteria that anchor surface proteins to |
| | peptidoglycans of the bacterial cell wall envelope. |
| | This involves a transpeptidation reaction in which |
| | the . . .; cd05826” |
| | /db_xref = “CDD: 99709” |
| Site | order(92, 114, 126, 128, 130, 222 . . . 223, 228) |
| | /site_type = “active” |
| | /db_xref = “CDD: 99709” |
| Site | order(130, 223) |
| | /site_type = “other” |
| | /note = “catalytic site” |
| | /db_xref = “CDD: 99709” |
| CDS | 1 . . . 244 |
| | /gene = “srtB” |
| | /locus_tag = “MW1017” |
| | /coded_by = “NC_003923.1: 1113163 . . . 1113897” |
| | /transl_table = 11 |
| | /db_xref = “GeneID: 1003129” |
| ORIGIN | |
| (SEQ ID NO; 4) | |
| 1 | mrmkrfltiv qillvviiii fgykivqtyi edkqeranye klqqkfqmlm skhqahvrpq |
| 61 | feslekinkd ivgwiklsgt slnypvlqgk tnhdylnldf erehrrkgsi fmdfrnelkn |
| 121 | lnhntilygh hvgdntmfdv ledylkqsfy ekhkiiefdn kygkyqlqvf sayktttkdn |
| 181 | yirtdfendq dyqqfldetk rksvinsdvn vtvkdrimtl stcedayset tkrivvvaki |
| 241 | ikvs |

1. An isolated nucleic acid comprising a nucleotide sequence encoding a polypeptide, the polypeptide comprising a eukaryotic signal sequence, a soluble sortase, and a transmembrane domain, wherein the signal sequence is capable of targeting the polypeptide for secretion by a eukaryotic host cell and the transmembrane domain is capable of anchoring the polypeptide in the plasma membrane of the host cell with the sortase exposed to the extracellular medium.

2. The nucleic acid of claim 1, wherein the sortase has sortase A catalytic activity.

3. The nucleic acid of claim 1 wherein the sortase is sortase A of S. aureus, or a catalytically active fragment, derivative, or variant thereof.

4. The nucleic acid of claim 2 wherein the sortase comprises residues 60-206 of sortase A of S. aureus (SEQ ID NO:2).

5. The nucleic acid of claim 1, wherein the transmembrane domain is capable of anchoring the polypeptide in the plasma membrane in a type II orientation.

6. The nucleic acid of claim 5, wherein the transmembrane domain is located N-terminal of the sortase.

7. The nucleic acid of claim 1, wherein the sortase has sortase B catalytic activity.

8. The nucleic acid of claim 7, wherein the sortase is sortase B of S. aureus, or a catalytically active fragment, derivative, or variant thereof.

9. The nucleic acid of claim 8, wherein sortase comprises residues 30-229 of sortase B of S. aureus (SEQ ID NO:4).

10. The nucleic acid of claim 9, wherein the transmembrane domain is capable of anchoring the polypeptide in the plasma membrane with a type I orientation.

11. The nucleic acid of claim 1, wherein the transmembrane domain is located C-terminal of the sortase.

12. The nucleic acid of claim 1, wherein the nucleotide sequence is operably linked to an expression control sequence.

13. The nucleic acid of claim 12, wherein the expression control sequence is a eukaryotic promoter.

14. The nucleic acid of claim 1, wherein the polypeptide further comprises an affinity tag.

15. The nucleic acid of claim 1, wherein the polypeptide further comprises a spacer peptide.

16. The nucleic acid of claim 15, wherein the spacer peptide is located between the soluble sortase and the transmembrane domain.

17. (canceled)

18. (canceled)

19. A recombinant polypeptide, comprising a eukaryotic signal sequence, a soluble sortase, and a transmembrane domain, wherein the signal sequence is capable of targeting the polypeptide for secretion by a eukaryotic host cell and the transmembrane domain is capable of anchoring the polypeptide in the plasma membrane of the host cell with the sortase exposed to the extracellular medium.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A composition comprising a conjugation substrate of the formula S-L-R, where S is the second sortase ligation sequence, L is an optional linker and R is the molecule of interest.

78. The composition of claim 77, wherein R is a water-soluble, non-peptidic polymer with an average molecular weight of about 200 to about 100,000 Daltons.

79. The composition of claim 78, wherein the polymer is a poly(ethylene glycol) (PEG) or a methoxypoly(ethylene glycol) (mPEG).

80. (canceled)

81. (canceled)

82. (canceled)

83. (canceled)

84. (canceled)

85. (canceled)

86. (canceled)

87. (canceled)

88. (canceled)
